SEQUENCE LISTING (18 sequences)

SEQ ID NO: 1 - DNA, 121 bp, Glycine max
  source 1..121: mol_type = genomic DNA
tggtgggtggtgacaatgaatcaggaacccaattggtggattgtggtgtggaagatgcaaygaagctttctatcagtgtccccacagcatcgcatccccacaacggtagatctagatctgg

SEQ ID NO: 2 - DNA, 121 bp, Glycine max
  source 1..121: mol_type = genomic DNA
ctctcagagatgggtaaccaattagtctcactacgcccctcagaagccacgggcaaaattrttacgtgggatggttcggtgcacgaaattgagcaatctttgacagtggcagagctgatgc

SEQ ID NO: 3 - DNA, 121 bp, Glycine max
  source 1..121: mol_type = genomic DNA
attagatgcctaagtagtgtcatttagatctttaaccttcattgtagtgttgcatattatdtttccattgtaatgatacaatcctaccttctaagggcattggatagaagactccaagaag

SEQ ID NO: 4 - DNA, 121 bp, Glycine max
  source 1..121: mol_type = genomic DNA
acacattaactttttatgatttatttgataatatatatgtttgagttaatgctgagtttamaatcagatggatcctaagtatttatggctttctatcattctctgaagcattcctgttgca

SEQ ID NO: 5 - DNA, 121 bp, Glycine max
  source 1..121: mol_type = genomic DNA
taggagtgttcgtacttcttccttagactagtgcacatgaattaaggattaatgcttaacrgatcgagataatttaaactaaaaaaattacagtatcttttttttaatatctcattattaa

SEQ ID NO: 6 - DNA, 121 bp, Glycine max
  source 1..121: mol_type = genomic DNA
aaaatgatttttgtgttatatcacaaaacttcaaggagtgttagtttagtttctttttttyaaggaacttagtttactcttttaaatattataagatgctctctctttagactcaaaggta

SEQ ID NO: 7 - DNA, 121 bp, Glycine max
  source 1..121: mol_type = genomic DNA
gaatcatcacaagttactgaacactagagtgaataatattttaaatgaaatgatcctaatycgcatcacttatgagtcacttgacgataacttaacccttaatctcgactaagaaacttat

SEQ ID NO: 8 - DNA, 121 bp, Glycine max
  source 1..121: mol_type = genomic DNA
atatccaaaaaatcaatagctttttgataaaccatttcttcgttcatccaaacaggatgtyataatatttatctagttcttcatttgctgatatgcatttggcagaataattgtcttcttt

SEQ ID NO: 9 - DNA, 121 bp, Glycine max
  source 1..121: mol_type = genomic DNA
ggagaagacattgatccttcttgttacgaacaagataccgcgggtctttcagaagaggaaktggaagagattagaaggcttcatgcaagtgacacggccattgacaaagagaaagactcaa

SEQ ID NO: 10 - DNA, 121 bp, Glycine max
  source 1..121: mol_type = genomic DNA
ataaaaaccatgcacttaaattgggtgcatcacgtgagtagccctttttttcagagaggaycaaatataaatattaaatatataaaattcaactaataattaaaagacttagtggttaatg

SEQ ID NO: 11 - DNA, 121 bp, Glycine max
  source 1..121: mol_type = genomic DNA
agtgattgtccgactaagatagagttgacttgacatattttactttttattccaatttctyttacttttttataaattgtattcctaatttttttatattttggcatattaaacaatgaca

SEQ ID NO: 12 - DNA, 121 bp, Glycine max
  source 1..121: mol_type = genomic DNA
ttctatctaaactaaccaaactcccattgctagccgaccttagagcatagttatcaaattygatttggaccaatcaggtcgacccattgaaccgagaactggggcaccaaattggctcaaa

SEQ ID NO: 13 - DNA, 121 bp, Glycine max
  source 1..121: mol_type = genomic DNA
aaaggtcacaattatttcgataaatatacctctgactgaataggtggtaggaaatcatggmaaacatatacaatgaataaaggatagcaagacaatacaaagtgatagaaagagatcaaac

SEQ ID NO: 14 - DNA, 121 bp, Glycine max
  source 1..121: mol_type = genomic DNA
cagaaaaaaagattaagctatgttgacccacaaaaatgccaaatacatgatactggaaagrctttaactctccttctctggttctattgtttgttgcgtcatagttcttgttggtgtatgc

SEQ ID NO: 15 - DNA, 121 bp, Glycine max
  source 1..121: mol_type = genomic DNA
accacaacaacttgacttgataaaagaatggcaaatgcatgaaaacatcaatttttgtaaytgtaccgttcacaatttctttattggaaacaatatggaataaagtagaccatgaatgcag

SEQ ID NO: 16 - DNA, 121 bp, Glycine max
  source 1..121: mol_type = genomic DNA
caaaccaaagtggacttgtgttacacgcttcatcatcactgcttccttcgaattccgaccrtagtattgaaagggggtcggatctgtggccaggtttcaataaccaaatgttgttggaaca

SEQ ID NO: 17 - DNA, 121 bp, Glycine max
  source 1..121: mol_type = genomic DNA
ttagaccctgctaaagaacaaagactccatcggtgttgggttgcagctgcgagaccaaagygtaagggacgtctttacagtattggagaccttgctcatatttataaatgtggagatgaga

SEQ ID NO: 18 - DNA, 121 bp, Glycine max
  source 1..121: mol_type = genomic DNA
tacattctcatttgcaagcttggacaaggcagagacatatgatcctaagtgggtaaatgcrcaggacctctgtgcccaaaagggttcaaaacttcaaggtggggttggaccatttgggctt

(19) United States
(12) Patent Application Publication
(10) Pub. No.: US 2024/0180093 A1
(43) Pub. Date: Jun. 6, 2024

(54) GENETIC MARKERS AND SOYBEAN PLANTS WITH INCREASED TOLERANCE TO DICAMBA

(71) Applicant: THE CURATORS OF THE UNIVERSITY OF MISSOURI, Columbia, MO (US)

(72) Inventors: Pengyin Chen, Collierville, TN (US); James Grover Shannon, Collierville, TN (US); Caio Canella Vieira, Portageville, MO (US); Henry Thien Nguyen, Columbia, MO (US)

(21) Appl. No.: 18/530,812

(22) Filed: Dec. 6, 2023

Related U.S. Application Data
(60) Provisional application No. 63/386,192, filed on Dec. 6, 2022.

(51) Int. Cl. A01H 1/04; A01H 1/00; A01H 5/10; A01H 6/54

(52) U.S. Cl. CPC A01H 1/045; A01H 1/123; A01H 5/10; A01H 6/542

(57) ABSTRACT

Markers associated with increased dicamba tolerance in soybeans are provided herein. Also provided are methods to identify plants having said markers and breeding methods to introduce said markers into other soybean plants, as well as methods for producing soybean plants having increased dicamba tolerance.

### CROSS REFERENCE TO RELATED APPLICATIONS

This application claims priority under 35 U.S.C. § 119 to provisional patent application U.S. Ser. No. 63/386,192, filed Dec. 6, 2022. The provisional patent application is herein incorporated by reference in its entirety, including without limitation, the specification, claims, and abstract, as well as any figures, tables, appendices, or drawings thereof.

### REFERENCE TO SEQUENCE LISTING SUBMITTED ELECTRONICALLY

The instant application contains a Sequence Listing which has been submitted electronically in XML format and is herein incorporated by reference in its entirety. Said XML copy, created on Dec. 6, 2023, is named “P14295US01_SequenceListing.xml” and is 18,731 bytes in size.

### TECHNICAL FIELD

The present disclosure relates generally to the field of plant breeding, and, more specifically, to methods for identifying and producing soybean varieties having increased tolerance to dicamba.

### BACKGROUND

Soybean (Glycine max (L.) Merr.) plays a multifaceted role in the global agricultural trade, economy, and food security due to its unique seed composition. As a major source of protein and vegetable oil, soybean is widely used in the food, feed, and biofuel industries. In the United States, approximately 95% of the soybean acreage is grown with genetically-engineered herbicide-tolerant cultivars, of which nearly 55% are grown using the dicamba-tolerance trait (DT, 3,6-dichloro-2-methoxybenzoic acid). Dicamba is a synthetic auxin (Group 4 herbicide) that triggers rapid and uncontrolled growth of the stems, petioles, and leaves, often leading to plant death in sensitive dicots. A distinguished characteristic of Group 4 herbicides is their high vapor pressure. Dicamba in specific has a vapor pressure of 2.0×10−5 mm HG at 25 C, which significantly increases the occurrence of off-target movement to adjacent fields. By comparison, glyphosate (N-(phosphonomethyl)glycine) has high water solubility and vapor pressure of 1.9×10−7 mm HG at 25° C.

The widespread adoption of DT cropping systems led to numerous cases of off-target damage to non-DT soybean as well as several other dicots plant species. Between 2017 and 2021, the Environmental Protection Agency (EPA) received over 10,500 reports of dicamba-related injuries in various non-DT vegetations in 29 of the 34 states where the use of dicamba on DT crops is authorized. Soybean is naturally sensitive to dicamba, and symptoms include crinkling and cupping of immature leaves, epinasty, plant height reduction, chlorosis, death of apical meristem, malformed pods, and ultimately yield reduction. The severity of the symptoms and yield loss differ based on the timing of exposure (growth stage), dosage, frequency, and duration of exposure. It is well known that the expression of a phenotype is a function of the genotype (G), the environment (E), and the differential phenotypic response of genotypes to different environments (G×E). However, information is lacking regarding the effect of different genetic backgrounds and identification of genomic regions affecting the severity of symptoms and yield loss caused by off-target dicamba in soybean.

With the advances in the availability of high-dimensional genomic data and comprehensive statistical models, genome-wide association studies (GWAS) have been largely used as a traditional approach to help reveal the underlying genetic basis of a trait of interest during the past decade. With several thousand to millions of single nucleotide polymorphisms (SNPs), GWAS captures significant associations between the trait of interest and molecular markers using linear or logistic regression analysis. In soybean, GWAS has unveiled the genetic architecture of multiple economic-important traits, including tolerance to biotic and abiotic stressors, seed composition, agronomic, physiology-efficient, as well as domestication-related traits. To date, GWAS has not been conducted to identify genomic regions associated with soybean tolerance to dicamba or other herbicides.

Thus, there exists a need in the art for the identification of significant marker-trait associations regulating the response of soybeans to off-target dicamba.

### SUMMARY

Markers associated with increased dicamba tolerance in soybean plants are provided, as are methods to produce soybean plants having at least one allele associated with dicamba tolerance. In some embodiments, the methods comprise providing a first soybean plant comprising at least one molecular marker associated with dicamba tolerance; providing a second soybean plant; crossing the first soybean plant with the second soybean plant to produce a population of soybean progeny plants; and selecting from said population of soybean progeny plants a soybean plant having at least one allele associated with dicamba tolerance. In some embodiments, the molecular marker is one or more of ss715635349, ss715605561, ss715593866, or a marker within 1 cM, 2 cM, or 5 cM thereof. In some embodiments, the molecular marker is a thymine (T) at position 61 of SEQ ID NO: 1, an adenine (A) at position 61 of SEQ ID NO: 2, and/or a T at position 61 of SEQ ID NO: 12. Plants and plant parts produced by said methods are also provided.

Methods of identifying and/or selecting a soybean plant having at least one allele associated with dicamba tolerance (such as that associated with off target dicamba) are also provided. In some embodiments, the methods comprise isolating a nucleic acid from a soybean plant; detecting in the nucleic acid the presence of at least one molecular marker associated with dicamba tolerance; and identifying and/or selecting a soybean plant based on the presence of at least one molecular marker associated with dicamba tolerance. In some embodiments, the molecular marker is one or more of ss715635349, ss715605561, ss715593866, or a marker within 1 cM, 2 cM, or 5 cM thereof. In some embodiments, the molecular marker is a thymine (T) at position 61 of SEQ ID NO: 1, an adenine (A) at position 61 of SEQ ID NO: 2, and/or a T at position 61 of SEQ ID NO: 12. In some embodiments, the identified and/or selected soybean plant exhibits increased tolerance to dicamba as compared to a control soybean plant lacking the at least one allele associated with dicamba tolerance. Plants and plant parts identified and/or selected by said methods are also provided. These plants may be crossed with a second soybean plant to produce progeny plants having at least one allele associated with dicamba tolerance.

Methods of identifying and/or selecting a soybean plant having increased tolerance to dicamba are also provided. In some embodiments, the method comprises screening a population of soybean plants with a molecular marker to determine if one or more soybean plants from the population comprises at least one allele associated with dicamba tolerance; and selecting and/or identifying from said population at least one soybean plant comprising the at least one allele associated with dicamba tolerance. In some embodiments, the molecular marker is one or more of ss715635349, ss715605561, ss715593866, or a marker within 1 cM, 2 cM, or 5 cM thereof. In some embodiments, the molecular marker is a thymine (T) at position 61 of SEQ ID NO: 1, an adenine (A) at position 61 of SEQ ID NO: 2, and/or a T at position 61 of SEQ ID NO: 12. In some embodiments, the method further comprises crossing the identified and/or selected soybean plant with a second soybean plant to obtain a progeny soybean plant. In some embodiments, the progeny soybean plants exhibit increased tolerance to dicamba as compared to a control soybean plant.

### BRIEF DESCRIPTION OF THE DRAWINGS

FIG. 1 shows the distribution of off-target dicamba damage scores at each testing environment and across environments from 2020-2021.

FIGS. 2A-2B show Manhattan plot highlighting significant marker-trait associations using the model that allows the inclusion of the population structure in interaction with environments (G×E) (2A) and BLINK (2B). Threshold of marker-trait association significance of approximately 4.0.

FIG. 3 shows distribution of off-target dicamba damage scores based on the allelic combination of SNPs ss715605561 (Chr. 10), ss715635349 (Chr. 19), and ss715632413 (Chr. 18). Favorable alleles were represented as 1 whereas unfavorable alleles were represented as 0.

FIG. 4 shows classification of genotypes based on the allelic combination of SNPs ss715605561 (Chr. 10), ss715635349 (Chr. 19), and ss715632413 (Chr. 18). Favorable alleles were represented as 1 whereas unfavorable alleles were represented as 0.

FIG. 5 shows machine learning-based GWAS pipeline scheme including feature dimension reduction (Partial Least Square), reduction of multicollinearity (Pairwise Pearson's Correlation), and identification of sets of SNPs conferring the highest classification accuracy (Forward stepwise selection loop using Random Forest and Support Vector Machine).

FIGS. 6A-6D show distribution of genotypes based on off-target dicamba response (tolerant, moderate, and susceptible) within each year and across all testing environments.

FIGS. 7A-7B show Manhattan plots highlighting the significant marker-trait associations identified using the BLINK (7A) and FarmCPU (7B) models. The threshold of marker-trait association significance was LOD>4.0.

FIG. 8 shows a Variable Importance in Projection (VIP)-based Manhattan plot of the 4,970 SNPs. The SNPs with VIP scores higher than 2.0 (above the dashed line) are highlighted in dark gray, and the 41 uncorrelated SNPs selected to be used in the ML-based GWAS are colored in light gray.

FIG. 9 shows a graphical confusion matrix based on the precision of each predicted class in the Random Forest and Support Vector Machine models.

FIG. 10 shows the overall prediction accuracy of each model's iteration from 1 to 2,000 SNPs as predictors. The decrease in prediction accuracy with the increment of the number of SNPs is a result of model overfitting.

### DETAILED DESCRIPTION

The present disclosure now will be described more fully with reference to the accompanying examples. The disclosure may be embodied in many different forms and should not be construed as limited to the embodiments set forth in this application; rather, these embodiments are provided so that this disclosure will satisfy applicable legal requirements.

Many modifications and other embodiments of the disclosure will come to mind to one skilled in the art to which this disclosure pertains, having the benefit of the teachings presented in the descriptions and the drawings herein. As a result, it is to be understood that the disclosure is not to be limited to the specific embodiments disclosed and that modifications and other embodiments are intended to be included within the scope of the appended claims. Although specific terms are used in the specification, they are used in a generic and descriptive sense only and not for purposes of limitation.

The following definitions and introductory matters are provided to facilitate an understanding of the present invention.

Numeric ranges recited within the specification, including ranges of “greater than,” “at least,” or “less than” a numeric value, are inclusive of the numbers defining the range and include each integer within the defined range.

The singular terms “a”, “an”, and “the” include plural referents unless context clearly indicates otherwise. Similarly, the word “or” is intended to include “and” unless the context clearly indicate otherwise. The word “or” means any one member of a particular list and also includes any combination of members of that list.

The term “allele” refers to one of two or more different nucleotide sequences that occur at a specific locus.

“Allele frequency” refers to the frequency (proportion or percentage) at which an allele is present at a locus within an individual, within a line, or within a population of lines. For example, for an allele “A”, diploid individuals of genotype “AA”, “Aa”, or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele.

An “amplicon” is an amplified nucleic acid, e.g., a nucleic acid that is produced by amplifying a template nucleic acid by any available amplification method (e.g., PCR, LCR, transcription, or the like).

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

An allele is “associated with” a trait when it is part of or linked to a DNA sequence or allele that affects the expression of the trait. The presence of the allele is an indicator of how the trait will be expressed.

“Backcrossing” refers to the process whereby hybrid progeny are repeatedly crossed back to one of the parents. In a backcrossing scheme, the “donor” parent refers to the parental plant with the desired gene/genes, locus/loci, or specific phenotype to be introgressed. The “recipient” parent (used one or more times) or “recurrent” parent (used two or more times) refers to the parental plant into which the gene or locus is being introgressed. For example, sec Ragot, M. et al. (1995) Marker-assisted backcrossing: a practical example, in Techniques et Utilisations des Marqueurs Moleculaires Les Colloques, Vol. 72, pp. 45-56, and Openshaw et al., (1994) Marker-assisted Selection in Backcross Breeding, Analysis of Molecular Marker Data, pp. 41-43. The initial cross gives rise to the F1 generation; the term “BC1” then refers to the second use of the recurrent parent, “BC2” refers to the third use of the recurrent parent, and so on.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval” designates a contiguous linear span of genomic DNA that resides in planta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

A “chromosome” is a single piece of coiled DNA containing many genes that act and move as a unity during cell division and therefore can be said to be linked. It can also be referred to as a “linkage group”.

The phrase “closely linked”, in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful in the present invention when they demonstrate a significant probability of co-segregation (linkage) with a desired trait. Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

The term “complement” refers to a nucleotide sequence that is complementary to a given nucleotide sequence, i.e. the sequences are related by the Watson-Crick base-pairing rules.

When referring to the relationship between two genetic elements, such as a genetic element contributing to a trait of interest and a proximal marker, “coupling” phase linkage indicates the state where the “favorable” allele at the genetic element contributing to increased resistance to anthracnose is physically associated on the same chromosome strand as the “favorable” allele of the respective linked marker locus. In coupling phase, both favorable alleles are inherited together by progeny that inherit that chromosome strand.

“Dicamba”, or 3,6-dichloro-2-methoxybenzoic acid, is a broad-spectrum herbicide primarily used in commercial settings to control weeds in grain crops and turf areas. Dicamba can have a tendency to spread from treated areas into neighboring areas, causing damage to unintended plants. This is referred to herein as “off-target dicamba”. “Dicamba tolerance” as used herein refers to soybean plants having increased tolerance to dicamba, including off-target dicamba, as compared to a control plant. In some embodiments, the control plant is a soybean plant lacking at least one allele associated with dicamba tolerance. In some embodiments, a dicamba tolerant plant is at least 10%, at least 20%, at least 30%, at least 40%, at least 50%, at least 75%, at least 90%, or at least 100% more tolerant to dicamba as compared to a control plant.

A plant referred to herein as “diploid” has two sets (genomes) of chromosomes.

A plant referred to herein as a “doubled haploid” is developed by doubling the haploid set of chromosomes (i.e., half the normal number of chromosomes). A doubled haploid plant has two identical sets of chromosomes, and all loci are considered homozygous.

“Elite line” means any line that has resulted from breeding and selection for superior agronomic performance. An “elite population” is an assortment of elite individuals or lines that can be used to represent the state of the art in terms of agronomically superior genotypes of a given crop species. Similarly, an “elite germplasm” or elite strain of germplasm is an agronomically superior germplasm.

A “favorable allele” is the allele at a particular locus that confers, or contributes to, an agronomically desirable phenotype, e.g., increased dicamba tolerance in a soybean plant, and that allows the identification of plants with that agronomically desirable phenotype. A favorable allele of a marker is a marker allele that segregates with the favorable phenotype.

“Fragment” is intended to mean a portion of a nucleotide sequence. Fragments can be used as hybridization probes or PCR primers using methods disclosed herein.

A “genetic map” is a description of genetic linkage relationships among loci on one or more chromosomes (or linkage groups) within a given species, generally depicted in a diagrammatic or tabular form. For each genetic map, distances between loci are measured by how frequently their alleles appear together in a population (their recombination frequencies). Alleles can be detected using DNA or protein markers, or observable phenotypes. A genetic map is a product of the mapping population, types of markers used, and the polymorphic potential of each marker between different populations. Genetic distances between loci can differ from one genetic map to another. However, information can be correlated from one map to another using common markers. One of ordinary skill in the art can use common marker positions to identify positions of markers and other loci of interest on each individual genetic map. The order of loci should not change between maps, although frequently there are small changes in marker orders due to e.g. markers detecting alternate duplicate loci in different populations, differences in statistical approaches used to order the markers, novel mutation or laboratory error.

A “genetic map location” is a location on a genetic map relative to surrounding genetic markers on the same linkage group where a specified marker can be found within a given species.

“Genetic mapping” is the process of defining the linkage relationships of loci through the use of genetic markers, populations segregating for the markers, and standard genetic principles of recombination frequency.

“Genetic markers” are nucleic acids that are polymorphic in a population and where the alleles of which can be detected and distinguished by one or more analytic methods, e.g., RFLP, AFLP, isozyme, SNP, SSR, and the like. The term also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD).

“Genetic recombination frequency” is the frequency of a crossing over event (recombination) between two genetic loci. Recombination frequency can be observed by following the segregation of markers and/or traits following meiosis.

“Genome” refers to the total DNA, or the entire set of genes, carried by a chromosome or chromosome set.

The term “genotype” is the genetic constitution of an individual (or group of individuals) at one or more genetic loci. Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome.

“Germplasm” refers to genetic material of or from an individual (e.g., a plant), a group of individuals (e.g., a plant line, variety or family), or a clone derived from a line, variety, species, or culture, or more generally, all individuals within a species or for several species. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leaf, stem, pollen, or cells, which can be cultured into a whole plant.

A plant referred to as “haploid” has a single set (genome) of chromosomes.

A “haplotype” is the genotype of an individual at a plurality of genetic loci, i.e. a combination of alleles. Typically, the genetic loci described by a haplotype are physically and genetically linked, i.e., on the same chromosome segment. The term “haplotype” can refer to alleles at a particular locus, or to alleles at multiple loci along a chromosomal segment.

The term “heterogeneity” is used to indicate that individuals within the group differ in genotype at one or more specific loci.

An individual is “heterozygous” if more than one allele type is present at a given locus (e.g., a diploid individual with one copy each of two different alleles).

The term “homogeneity” indicates that members of a group have the same genotype at one or more specific loci.

An individual is “homozygous” if the individual has only one type of allele at a given locus (e.g., a diploid individual has a copy of the same allele at a locus for each of two homologous chromosomes).

The term “hybrid” refers to the progeny obtained between the crossing of at least two genetically dissimilar parents.

“Hybridization” or “nucleic acid hybridization” refers to the pairing of complementary RNA and DNA strands as well as the pairing of complementary DNA single strands.

The term “hybridize” means to form base pairs between complementary regions of nucleic acid strands.

The term “inbred” refers to a line that has been bred for genetic homogeneity.

The term “introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. In any case, offspring comprising the desired allele can be repeatedly backcrossed to a line having a desired genetic background and selected for the desired allele, to result in the allele becoming fixed in a selected genetic background.

The process of “introgressing” is often referred to as “backcrossing” when the process is repeated two or more times.

A “line” or “strain” is a group of individuals of identical parentage that are generally inbred to some degree and that are generally homozygous and homogeneous at most loci (isogenic or near isogenic). A “subline” refers to an inbred subset of descendants that are genetically distinct from other similarly inbred subsets descended from the same progenitor.

As used herein, the term “linkage” is used to describe the degree with which one marker locus is associated with another marker locus or some other locus. The linkage relationship between a molecular marker and a locus affecting a phenotype is given as a “probability” or “adjusted probability”. Linkage can be expressed as a desired limit or range. For example, in some embodiments, any marker is linked (genetically and physically) to any other marker when the markers are separated by less than 50, 40, 30, 25, 20, or 15 map units (or cM). In some aspects, it is advantageous to define a bracketed range of linkage, for example, between 10 and 20 cM, between 10 and 30 cM, or between 10 and 40 cM. The more closely a marker is linked to a second locus, the better an indicator for the second locus that marker becomes. Thus, “closely linked loci” such as a marker locus and a second locus display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “in proximity to” each other. Since one cM is the distance between two markers that show a 1% recombination frequency, any marker is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype. A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score).

Linkage disequilibrium is most commonly assessed using the measure r², which is calculated using the formula described by Hill, W. G. and Robertson, A, Theor. Appl. Genet. 38:226-231(1968). When r²=1, complete linkage disequilibrium exists between the two marker loci, meaning that the markers have not been separated by recombination and have the same allele frequency. The r² value will be dependent on the population used. Values for r² above ⅓ indicate sufficiently strong linkage disequilibrium to be useful for mapping (Ardlie et al., Nature Reviews Genetics 3:299-309 (2002)). Hence, alleles are in linkage disequilibrium when r² values between pairwise marker loci are greater than or equal to 0.33, 0.4, 0.5, 0.6, 0.7, 0.8, 0.9, or 1.0.

A “locus” is a position on a chromosome, e.g. where a nucleotide, gene, sequence, or marker is located.

The “logarithm of odds (LOD) value” or “LOD score” (Risch, Science 255:803-804 (1992)) is used in genetic interval mapping to describe the degree of linkage between two marker loci. A LOD score of three between two markers indicates that linkage is 1000 times more likely than no linkage, while a LOD score of two indicates that linkage is 100 times more likely than no linkage. LOD scores greater than or equal to two may be used to detect linkage. LOD scores can also be used to show the strength of association between marker loci and quantitative traits in “quantitative trait loci” mapping. In this case, the LOD score's size is dependent on the closeness of the marker locus to the locus affecting the quantitative trait, as well as the size of the quantitative trait effect.

A “marker” is a means of finding a position on a genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), or a simply inherited phenotype (such as the ‘waxy’ phenotype). A DNA marker can be developed from genomic nucleotide sequence or from expressed nucleotide sequences (e.g., from a spliced RNA or a cDNA). Depending on the DNA marker technology, the marker will consist of complementary primers flanking the locus and/or complementary probes that hybridize to polymorphic alleles at the locus. A DNA marker, or a genetic marker, can also be used to describe the gene, DNA sequence or nucleotide on the chromosome itself (rather than the components used to detect the gene or DNA sequence) and is often used when that DNA marker is associated with a particular trait in human genetics (e.g. a marker for breast cancer). The term marker locus is the locus (gene, sequence or nucleotide) that the marker detects.

Markers that detect genetic polymorphisms between members of a population are well-established in the art. Markers can be defined by the type of polymorphism that they detect and also the marker technology used to detect the polymorphism. Marker types include but are not limited to, e.g., detection of restriction fragment length polymorphisms (RFLP), detection of isozyme markers, randomly amplified polymorphic DNA (RAPD), amplified fragment length polymorphisms (AFLPs), detection of simple sequence repeats (SSRs), detection of amplified variable sequences of the plant genome, detection of self-sustained sequence replication, or detection of single nucleotide polymorphisms (SNPs). SNPs can be detected e.g. via DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype. Haplotypes tend to be more informative (detect a higher level of polymorphism) than SNPs.

A “marker allele”, alternatively an “allele of a marker locus”, can refer to one of a plurality of polymorphic nucleotide sequences found at a marker locus in a population.

“Marker assisted selection” (or MAS) is a process by which individual plants are selected based on marker genotypes.

A “marker haplotype” refers to a combination of alleles at a marker locus.

A “marker locus” is a specific chromosome location in the genome of a species where a specific marker can be found. A marker locus can be used to track the presence of a second linked locus, e.g., one that affects the expression of a phenotypic trait. For example, a marker locus can be used to monitor segregation of alleles at a genetically or physically linked locus.

A “marker probe” is a nucleic acid sequence or molecule that can be used to identify the presence of a marker locus, e.g., a nucleic acid probe that is complementary to a marker locus sequence, through nucleic acid hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e., genotype) the particular allele that is present at a marker locus.

The term “molecular marker” may be used to refer to a genetic marker, as defined above, or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g. SNP technology is used in the examples provided herein.

An allele “negatively” correlates with a trait when it is linked to it and when presence of the allele is an indicator that a desired trait or trait form will not occur in a plant comprising the allele.

“Nucleotide sequence”, “polynucleotide”, “nucleic acid sequence”, and “nucleic acid fragment” are used interchangeably and refer to a polymer of RNA or DNA that is single or double-stranded, optionally containing synthetic, non-natural or altered nucleotide bases. A “nucleotide” is a monomeric unit from which DNA or RNA polymers are constructed, and consists of a purine or pyrimidine base, a pentose, and a phosphoric acid group. Nucleotides (usually found in their 5′ monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The term “phenotype”, “phenotypic trait”, or “trait” can refer to the observable expression of a gene or series of genes. The phenotype can be observable to the naked eye, or by any other means of evaluation known in the art, e.g., weighing, counting, measuring (length, width, angles, etc.), microscopy, biochemical analysis, or an electromechanical assay. In some cases, a phenotype is directly controlled by a single gene or genetic locus, i.e., a “single gene trait” or a “simply inherited trait”. In the absence of large levels of environmental variation, single gene traits can segregate in a population to give a “qualitative” or “discrete” distribution, i.e. the phenotype falls into discrete classes. In other cases, a phenotype is the result of several genes and can be considered a “multigenic trait” or a “complex trait”. Multigenic traits segregate in a population to give a “quantitative” or “continuous” distribution, i.e. the phenotype cannot be separated into discrete classes. Both single gene and multigenic traits can be affected by the environment in which they are being expressed, but multigenic traits tend to have a larger environmental component.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination (that can vary in different populations).

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant.

A “polymorphism” is a variation in the DNA between two or more individuals within a population. A polymorphism preferably has a frequency of at least 1% in a population. A useful polymorphism can include a single nucleotide polymorphism (SNP), a simple sequence repeat (SSR), or an insertion/deletion polymorphism, also referred to herein as an “indel”.

An allele “positively” correlates with a trait when it is linked to it and when presence of the allele is an indicator that the desired trait or trait form will occur in a plant comprising the allele.

The “probability value” or “p-value” is the statistical likelihood that the particular combination of a phenotype and the presence or absence of a particular marker allele is random. Thus, the lower the probability score, the greater the likelihood that a locus and a phenotype are associated. The probability score can be affected by the proximity of the first locus (usually a marker locus) and the locus affecting the phenotype, plus the magnitude of the phenotypic effect (the change in phenotype caused by an allele substitution). In some aspects, the probability score is considered “significant” or “nonsignificant”. In some embodiments, a probability score of 0.05 (p=0.05, or a 5% probability) of random assortment is considered a significant indication of association. However, an acceptable probability can be any probability of less than 50% (p=0.5). For example, a significant probability can be less than 0.25, less than 0.20, less than 0.15, less than 0.1, less than 0.05, less than 0.01, or less than 0.001.

A “production marker” or “production SNP marker” is a marker that has been developed for high-throughput purposes. Production SNP markers are developed to detect specific polymorphisms and are designed for use with a variety of chemistries and platforms.

The term “progeny” refers to the offspring generated from a cross. A “progeny plant” is a plant generated from a cross between two plants.

The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question. An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more polymorphic markers. A haplotype can be defined by the unique fingerprint of alleles at each marker within the specified window.

A “reference sequence” or a “consensus sequence” is a defined sequence used as a basis for sequence comparison. The reference sequence for a PHM marker is obtained by sequencing a number of lines at the locus, aligning the nucleotide sequences in a sequence alignment program (e.g. Sequencher), and then obtaining the most common nucleotide sequence of the alignment. Polymorphisms found among the individual sequences are annotated within the consensus sequence. A reference sequence is not usually an exact copy of any individual DNA sequence, but represents an amalgam of available sequences and is useful for designing primers and probes to polymorphisms within the sequence.

In “repulsion” phase linkage, the “favorable” allele at the locus of interest is physically linked with an “unfavorable” allele at the proximal marker locus, and the two “favorable” alleles are not inherited together (i.e., the two loci are “out of phase” with each other).

As used herein, the term “soybean” refers to a plant, and any part thereof, of the genus Glycine including, but not limited to Glycine max.

The term “soybean plant” includes whole soybean plants, soybean plant cells, soybean plant protoplast, soybean plant cell or soybean tissue culture from which soybean plants can be regenerated, soybean plant calli, soybean plant clumps and soybean plant cells that are intact in soybean plants or parts of soybean plants, such as soybean seeds, soybean flowers, soybean cotyledons, soybean leaves, soybean stems, soybean buds, soybean roots, soybean root tips and the like.

A “topcross test” is a test performed by crossing each individual (e.g. a selection, inbred line, clone or progeny individual) with the same pollen parent or “tester”, usually a homozygous line.

The phrase “under stringent conditions” refers to conditions under which a probe or polynucleotide will hybridize to a specific nucleic acid sequence, typically in a complex mixture of nucleic acids, but to essentially no other sequences. Stringent conditions are sequence-dependent and will be different in different circumstances. Longer sequences hybridize specifically at higher temperatures. Generally, stringent conditions are selected to be about 5-10° C. lower than the thermal melting point (Tm) for the specific sequence at a defined ionic strength pH. The Tm is the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3, and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions are often: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C., depending on primer length. Additional guidelines for determining hybridization parameters are provided in numerous references.

An “unfavorable allele” of a marker is a marker allele that segregates with the unfavorable plant phenotype, therefore providing the benefit of identifying plants that can be removed from a breeding program or planting.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989.

### Dicamba Tolerance in Soybeans

Widespread adoption of dicamba tolerant (DT) production systems has frequently resulted in yield losses in non-DT soybean genotypes from off-target dicamba exposure. Environmental conditions that exacerbate the likelihood of off-target movement are often observed in soybean-producing regions during the growing season, hence the reports of damage in most states where the over-the-top use of dicamba is authorized. Soybean is highly sensitive to dicamba exposure, critically compromising the yield and quality of non-DT genetically engineered and non-GMO growing systems. Identification of genetic sources of tolerance and genomic regions conferring higher tolerance to off-target dicamba in non-DT soybean genotypes may sustain and improve other non-DT soybean production systems, including the growing niche markets of organic and conventional soybean. The present disclosure identifies genetic sources for improved tolerance to off-target dicamba. The genetic architecture of tolerance is complex and regulated by multiple small and large effect loci. However, ss715593866 is a major effect SNP and resulted in high classification accuracies. Candidate genes with biological functions associated with herbicide detoxification in plants were co-localized with significant minor and major effect SNPs. Two genomic regions on Chrs. 10 (ss715605561) and 19 (ss715635349) were identified that may be directly associated with the ability of soybean to detoxify dicamba and/or transport non-phytotoxic dicamba metabolites into the vacuole. Furthermore, three significant SNPs (ss715605561 (Chr. 10), ss715635349 (Chr. 19), and ss715632413 (Chr. 18)) can be used to accurately distinguish between tolerant and susceptible genotypes. With the advancements in targeted gene-editing techniques, the present disclosure may facilitate identifying and developing conventional soybean cultivars with improved tolerance to off-target dicamba as well as other synthetic auxin herbicides.

### Genetic Mapping

It has been recognized for quite some time that specific genetic loci correlating with particular traits can be mapped in an organism's genome. The plant breeder can advantageously use molecular markers to identify desired individuals by detecting marker alleles that show a statistically significant probability of co-segregation with a desired phenotype, manifested as linkage disequilibrium. By identifying a molecular marker or clusters of molecular markers that co-segregate with a trait of interest, the breeder is able to rapidly select a desired phenotype by selecting for the proper molecular marker allele (a process called marker-assisted selection).

A variety of methods well known in the art are available for detecting molecular markers or clusters of molecular markers that co-segregate with a trait of interest, such as dicamba tolerance in soybeans. The basic idea underlying these methods is the detection of markers, for which alternative genotypes (or alleles) have significantly different average phenotypes. Thus, one makes a comparison among marker loci of the magnitude of difference among alternative genotypes (or alleles) or the level of significance of that difference. Trait genes are inferred to be located nearest the marker(s) that have the greatest associated genotypic difference. Two such methods used to detect trait loci of interest are: 1) Population-based association analysis and 2) Traditional linkage analysis.

In a population-based association analysis, lines are obtained from pre-existing populations with multiple founders, e.g. elite breeding lines. Population-based association analyses rely on linkage disequilibrium (LD) and the idea that in an unstructured population, only correlations between genes controlling a trait of interest and markers closely linked to those genes will remain after so many generations of random mating. In reality, most pre-existing populations have population substructure. Thus, the use of a structured association approach helps to control population structure by allocating individuals to populations using data obtained from markers randomly distributed across the genome, thereby minimizing disequilibrium due to population structure within the individual populations (also called subpopulations). The phenotypic values are compared to the genotypes (alleles) at each marker locus for each line in the subpopulation. A significant marker-trait association indicates the close proximity between the marker locus and one or more genetic loci that are involved in the expression of that trait.

The same principles underlie traditional linkage analysis; however, linkage disequilibrium is generated by creating a population from a small number of founders. The founders are selected to maximize the level of polymorphism within the constructed population, and polymorphic sites are assessed for their level of cosegregation with a given phenotype. A number of statistical methods have been used to identify significant marker-trait associations. One such method is an interval mapping approach (Lander and Botstein, Genetics 121:185-199 (1989), in which each of many positions along a genetic map (say at 1 cM intervals) is tested for the likelihood that a gene controlling a trait of interest is located at that position. The genotype/phenotype data are used to calculate for each test position a LOD score (log of likelihood ratio). When the LOD score exceeds a threshold value, there is significant evidence for the location of a gene controlling the trait of interest at that position on the genetic map (which will fall between two particular marker loci).

The present invention provides soybean marker loci that demonstrate statistically significant co-segregation with dicamba tolerance as determined by the linkage analysis methods described in the Examples. Detection of these loci or additional linked loci can be used in marker assisted soybean breeding programs to produce plants with increased dicamba tolerance.

### Markers and Linkage Relationships

A common measure of linkage is the frequency with which traits cosegregate. This can be expressed as a percentage of cosegregation (recombination frequency) or in centiMorgans (cM). The cM is a unit of measure of genetic recombination frequency. One cM is equal to a 1% chance that a trait at one genetic locus will be separated from a trait at another locus due to crossing over in a single generation (meaning the traits segregate together 99% of the time). Because chromosomal distance is approximately proportional to the frequency of crossing over events between traits, there is an approximate physical distance that correlates with recombination frequency.

Marker loci are themselves traits and can be assessed according to standard linkage analysis by tracking the marker loci during segregation. Thus, one cM is equal to a 1% chance that a marker locus will be separated from another locus, due to crossing over in a single generation.

The closer a marker is to a gene controlling a trait of interest, the more effective and advantageous that marker is as an indicator for the desired trait. Closely linked loci display an inter-locus cross-over frequency of about 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a target locus) display a recombination frequency of about 1% or less, e.g., about 0.75% or less, more preferably about 0.5% or less, or yet more preferably about 0.25% or less. Thus, the loci are about 10 cM, 9 cM, 8 cM, 7 cM, 6 cM, 5 cM, 4 cM, 3 cM, 2 cM, 1 cM, 0.75 cM, 0.5 cM or 0.25 cM or less apart. Put another way, two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are said to be “proximal to” each other.

Although particular marker alleles can co-segregate with increased or decreased dicamba tolerance, it is important to note that the marker locus is not necessarily responsible for the expression of the dicamba tolerance phenotype. For example, it is not a requirement that the marker polynucleotide sequence be part of a gene that is responsible for the phenotype (for example, is part of the gene open reading frame). The association between a specific marker allele and a trait is due to the original “coupling” linkage phase between the marker allele and the allele in the ancestral soybean line from which the allele originated. Eventually, with repeated recombination, crossing over events between the marker and genetic locus can change this orientation. For this reason, the favorable marker allele may change depending on the linkage phase that exists within the parent having the favorable trait that is used to create segregating populations. This does not change the fact that the marker can be used to monitor segregation of the phenotype. It only changes which marker allele is considered favorable in a given segregating population.

Methods presented herein include detecting the presence of one or more marker alleles associated with increased dicamba tolerance in a soybean plant and then identifying and/or selecting soybean plants that have favorable alleles at those marker loci, or detecting the presence of a marker allele associated with decreased dicamba tolerance and then identifying and/or counterselecting soybean plants that have unfavorable alleles. Markers have been identified herein as being associated with dicamba tolerance in soybeans and hence can be used to identify and select soybean plants having increased dicamba tolerance. Any marker within 20 cM, 15 cM, 10 cM, 9 cM, 8 cM, 7 cM, 6 cM, 5 cM, 4 cM, 3 cM, 2 cM, 1 cM, 0.9 cM, 0.8 cM, 0.7 cM, 0.6 cM, 0.5 cM, 0.4 cM, 0.3 cM, 0.2 cM, 0.1 cM or less of any of the markers identified herein could also be used to identify and select soybean plants with increased dicamba tolerance. Any marker allele linked to and associated with the favorable alleles of the markers listed herein can be used for detection purposes in the identification and/or selection of plants with increased dicamba tolerance.

The present disclosure provides SNP markers and/or combinations of SNP markers that can be used in various aspects of the presently disclosed subject matter as set forth herein.

Thus, the SNP markers provided herein can be used for detecting the presence of one or more alleles associated with dicamba tolerance in a soybean plant or germplasm, and can therefore be used in methods involving marker-assisted breeding and selection of soybean plants having increased dicamba tolerance/soybean plants having one or more alleles associated with increased dicamba tolerance.

Accordingly, SNP molecular markers associated with dicamba tolerance alleles are identified herein. In some embodiments, the molecular marker is one or more of ss715635349, ss715605561, ss715609879, ss715632413, ss715622838, ss715619759, ss715590768, ss715592527, ss715632432, ss715632412, ss715592728, ss715593866, ss715594836, ss715600920, ss715604850, ss715608720, ss715633252, ss715635454, or a marker within 1 cM, 2 cM, or 5 cM thereof. In some embodiments, the molecular marker is one or more of ss715635349, ss715605561, ss715593866, or a marker within 1 cM, 2 cM, or 5 cM thereof. In some embodiments, the molecular marker is one or more of ss715605561, ss715635349, ss715632413, or a marker within 1 cM, 2 cM, or 5 cM thereof.

In some embodiments, the molecular marker is a SNP at position 61 of one or more of SEQ ID NOs: 1-18. In some embodiments, the marker is a thymine (T) at position 61 of SEQ ID NO:1, an adenine (A) at position 61 of SEQ ID NO: 2, a T at position 61 of SEQ ID NO: 3, an A at position 61 of SEQ ID NO: 4, a guanine (G) at position 61 of SEQ ID NO: 5, a T at position 61 of SEQ ID NO: 6, a T at position at position 61 of SEQ ID NO: 7, a cytosine (C) at position 61 of SEQ ID NO: 8, a G at position 61 of SEQ ID NO: 9, a Cat position 61 of SEQ ID NO: 10, a T at position 61 of SEQ ID NO: 11, a T at position 61 of SEQ ID NO: 12, an A at position 61 of SEQ ID NO: 13, an A at position 61 of SEQ ID NO: 14, a C at position 61 of SEQ ID NO: 15, an A at position 61 of SEQ ID NO: 16, a T at position 61 of SEQ ID NO: 17, and/or an A at position 61 of SEQ ID NO: 18. In some embodiments, the molecular marker is a thymine (T) at position 61 of SEQ ID NO: 1, an adenine (A) at position 61 of SEQ ID NO: 2, and/or a T at position 61 of SEQ ID NO: 12.

In some embodiments, as described herein, a combination of SNPs can be used to detect the presence of an allele associated with dicamba tolerance.

In further embodiments, a marker of this disclosure can include any marker linked to the aforementioned markers. Linked markers may be determined, for example, by using resources available on the SoyBase internet resource (soybase.org).

### Marker Assisted Selection

Molecular markers can be used in a variety of plant breeding applications (e.g. see Staub et al. (1996) Hortscience 31: 729-741; Tanksley (1983) Plant Molecular Biology Reporter. 1: 3-8). One of the main areas of interest is to increase the efficiency of backcrossing and introgressing genes using marker-assisted selection. A molecular marker that demonstrates linkage with a locus affecting a desired phenotypic trait provides a useful tool for the selection of the trait in a plant population. This is particularly true where the phenotype is hard to assay. Since DNA marker assays are less laborious and take up less physical space than field phenotyping, much larger populations can be assayed, increasing the chances of finding a recombinant with the target segment from the donor line moved to the recipient line. The closer the linkage, the more useful the marker, as recombination is less likely to occur between the marker and the gene causing the trait, which can result in false positives. Having flanking markers decreases the chances that false positive selection will occur as a double recombination event would be needed. The ideal situation is to have a marker in the gene itself, so that recombination cannot occur between the marker and the gene. Such a marker is called a ‘perfect marker’.

When a gene is introgressed by marker assisted selection, it is not only the gene that is introduced but also the flanking regions (Gepts. (2002). Crop Sci; 42: 1780-1790). This is referred to as “linkage drag.” In the case where the donor plant is highly unrelated to the recipient plant, these flanking regions carry additional genes that may code for agronomically undesirable traits. This “linkage drag” may also result in reduced yield or other negative agronomic characteristics even after multiple cycles of backcrossing into the elite soybean line. This is also sometimes referred to as “yield drag.” The size of the flanking region can be decreased by additional backcrossing, although this is not always successful, as breeders do not have control over the size of the region or the recombination breakpoints (Young et al. (1998) Genetics 120:579-585). In classical breeding it is usually only by chance that recombinations are selected that contribute to a reduction in the size of the donor segment (Tanksley et al. (1989). Biotechnology 7: 257-264). Even after 20 backcrosses in backcrosses of this type, one may expect to find a sizeable piece of the donor chromosome still linked to the gene being selected. With markers however, it is possible to select those rare individuals that have experienced recombination near the gene of interest. In 150 backcross plants, there is a 95% chance that at least one plant will have experienced a crossover within 1 cM of the gene. Markers will allow unequivocal identification of those individuals. With one additional backcross of 300 plants, there would be a 95% chance of a crossover within 1 cM of the other side of the gene, generating a segment around the target gene of less than 2 cM. This can be accomplished in two generations with markers, while it would have required on average 100 generations without markers (See Tanksley et al., supra). When the exact location of a gene is known, flanking markers surrounding the gene can be utilized to select for recombinations in different population sizes. For example, in smaller population sizes, recombinations may be expected further away from the gene, so more distal flanking markers would be required to detect the recombination.

The key components to the implementation of marker assisted selection are: (i) Defining the population within which the marker-trait association will be determined, which can be a segregating population, or a random or structured population; (ii) monitoring the segregation or association of polymorphic markers relative to the trait, and determining linkage or association using statistical methods; (iii) defining a set of desirable markers based on the results of the statistical analysis, and (iv) the use and/or extrapolation of this information to the current set of breeding germ plasm to enable marker-based selection decisions to be made. The markers described in this disclosure, as well as other marker types such as SSRs and FLPs, can be used in marker assisted selection protocols.

SSRs can be defined as relatively short runs of tandemly repeated DNA with lengths of 6 bp or less (Tautz (1989) Nucleic Acid Research 17: 6463-6471; Wang et al. (1994) Theoretical and Applied Genetics, 88:1-6). Polymorphisms arise due to variation in the number of repeat units, probably caused by slippage during DNA replication (Levinson and Gutman (1987) Mol Biol Evol 4: 203-221). The variation in repeat length may be detected by designing PCR primers to the conserved non-repetitive flanking regions (Weber and May (1989) Am J Hum Genet. 44:388-396). SSRs are highly suited to mapping and marker assisted selection as they are multi-allelic, codominant, reproducible and amenable to high throughput automation (Rafalski et al. (1996) Generating and using DNA markers in plants. In: Non-mammalian genomic analysis: a practical guide. Academic press. pp 75-135).

Various types of SSR markers can be generated, and SSR profiles can be obtained by gel electrophoresis of the amplification products. Scoring of marker genotype is based on the size of the amplified fragment. Various types of FLP markers can also be generated. Most commonly, amplification primers are used to generate fragment length polymorphisms. Such FLP markers are in many ways similar to SSR markers, except that the region amplified by the primers is not typically a highly repetitive region. Still, the amplified region, or amplicon, will have sufficient variability among germplasm, often due to insertions or deletions, such that the fragments generated by the amplification primers can be distinguished among polymorphic individuals, and such indels are known to occur frequently in soybeans (Evans et al. PLos One (2013). 8 (11): e79192).

SNP markers detect single base pair nucleotide substitutions. Of all the molecular marker types, SNPs are the most abundant, thus having the potential to provide the highest genetic map resolution (PLos One (2013). 8 (11): e79192). SNPs can be assayed at an even higher level of throughput than SSRs, in a so-called ‘ultra-high-throughput’ fashion, as they do not require large amounts of DNA and automation of the assay may be straight-forward. SNPs also have the promise of being relatively low-cost systems. These three factors together make SNPs highly attractive for use in marker assisted selection. Several methods are available for SNP genotyping, including but not limited to, hybridization, primer extension, oligonucleotide ligation, nuclease cleavage, minisequencing and coded spheres. Such methods have been reviewed in: Gut (2001) Hum Mutat 17 pp. 475-492; Shi (2001) Clin Chem 47, pp. 164-172; Kwok (2000) Pharmacogenomics 1, pp. 95-100; and Bhattramakki and Rafalski (2001) Discovery and application of single nucleotide polymorphism markers in plants. In: R. J. Henry, Ed, Plant Genotyping: The DNA Fingerprinting of Plants, CABI Publishing, Wallingford. A wide range of commercially available technologies utilize these and other methods to interrogate SNPs including Masscode™ (Qiagen), INVADER®. (Third Wave Technologies) and Invader PLUS®, SNAPSHOT®. (Applied Biosystems), TAQMAN®. (Applied Biosystems) and BEADARRAYS®. (Illumina).

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes can be more informative than single SNPs and can be more descriptive of any particular genotype. For example, a single SNP may be allele ‘T’ for a specific line or variety with early maturity, but the allele ‘T’ might also occur in the soybean breeding population being utilized for recurrent parents. In this case, a haplotype, e.g. a combination of alleles at linked SNP markers, may be more informative. Once a unique haplotype has been assigned to a donor chromosomal region, that haplotype can be used in that population or any subset thereof to determine whether an individual has a particular gene. See, for example, WO2003054229. Using automated high throughput marker detection platforms known to those of ordinary skill in the art makes this process highly efficient and effective.

In addition to SSR's, FLPs and SNPs, as described above, other types of molecular markers are also widely used, including but not limited to expressed sequence tags (ESTs), SSR markers derived from EST sequences, randomly amplified polymorphic DNA (RAPD), and other nucleic acid based markers.

Isozyme profiles and linked morphological characteristics can, in some cases, also be indirectly used as markers. Even though they do not directly detect DNA differences, they are often influenced by specific genetic differences. However, markers that detect DNA variation are far more numerous and polymorphic than isozyme or morphological markers (Tanksley (1983) Plant Molecular Biology Reporter 1:3-8).

Sequence alignments or contigs may also be used to find sequences upstream or downstream of the specific markers listed herein. These new sequences, close to the markers described herein, are then used to discover and develop functionally equivalent markers. For example, different physical and/or genetic maps are aligned to locate equivalent markers not described within this disclosure but that are within similar regions. These maps may be within the soybean species, or even across other species that have been genetically or physically aligned with soybean, such as maize, rice, wheat, or barley.

In general, marker assisted selection uses polymorphic markers that have been identified as having a significant likelihood of co-segregation with a phenotype, such as dicamba tolerance in soybean. Such markers are presumed to map near a gene or genes that regulate dicamba tolerance in a soybean plant, and are considered indicators for the desired trait, or markers. Plants are tested for the presence of a desired allele in the marker, and plants containing a desired genotype at one or more loci are expected to transfer the desired genotype, along with a desired phenotype, to their progeny. Thus, soybean plants with increased dicamba tolerance can be selected for by detecting one or more marker alleles, and in addition, progeny plants derived from those plants can also be selected. Hence, a plant containing a desired genotype in a given chromosomal region is obtained and then crossed to another plant. The progeny of such a cross would then be evaluated genotypically using one or more markers and the progeny plants with the same genotype in a given chromosomal region would then be selected as exhibiting increased dicamba tolerance.

Markers were identified from linkage mapping analysis as being associated with dicamba tolerance. The SNPs identified herein could be used alone or in combination (i.e. a SNP haplotype) to select for plants having a favorable QTL allele (i.e. associated with increased dicamba tolerance). The skilled artisan would expect that there might be additional polymorphic sites at marker loci in and around the markers identified herein, wherein one or more polymorphic sites is in linkage disequilibrium with an allele at one or more of the polymorphic sites in the haplotype and thus could be used in a marker assisted selection program to introgress a QTL allele of interest. Two particular alleles at different polymorphic sites are said to be in linkage disequilibrium if the presence of the allele at one of the sites tends to predict the presence of the allele at the other site on the same chromosome (Stevens, Mol. Diag. 4:309-17 (1999)).

The skilled artisan would understand that allelic frequency (and hence, haplotype frequency) can differ from one germplasm pool to another. Germplasm pools vary due to maturity differences, heterotic groupings, geographical distribution, etc. As a result, SNPs and other polymorphisms may not be informative in some germplasm pools.

### Use in Breeding Methods

The plants of the disclosure may be used in a plant breeding program. The goal of plant breeding is to combine, in a single variety or hybrid, various desirable traits. For field crops, these traits may include, for example, resistance to diseases and insects, tolerance to heat and drought, tolerance to herbicides or pesticides, tolerance to chilling or freezing, reduced time to crop maturity, greater yield and better agronomic quality. With mechanical harvesting of many crops, uniformity of plant characteristics such as germination and stand establishment, growth rate, maturity and plant and car height is desirable. Traditional plant breeding is an important tool in developing new and improved commercial crops. This disclosure encompasses methods for producing a plant by crossing a first parent plant with a second parent plant wherein one or both of the parent plants is a plant displaying a phenotype as described herein.

Plant breeding techniques known in the art and used in a plant breeding program include, but are not limited to, recurrent selection, bulk selection, mass selection, backcrossing, pedigree breeding, open pollination breeding, restriction fragment length polymorphism enhanced selection, genetic marker enhanced selection, doubled haploids and transformation. Often combinations of these techniques are used.

The development of hybrids in a plant breeding program requires, in general, the development of homozygous inbred lines, the crossing of these lines and the evaluation of the crosses. There are many analytical methods available to evaluate the result of a cross. The oldest and most traditional method of analysis is the observation of phenotypic traits. Alternatively, the genotype of a plant can be examined.

A genetic trait which has been engineered into a particular plant using transformation techniques can be moved into another line using traditional breeding techniques that are well known in the plant breeding arts. For example, a backcrossing approach is commonly used to move a transgene from a transformed plant to an elite inbred line and the resulting progeny would then comprise the transgene(s). Also, if an inbred line was used for the transformation, then the transgenic plants could be crossed to a different inbred in order to produce a transgenic hybrid plant. As used herein, “crossing” can refer to a simple X by Y cross or the process of backcrossing, depending on the context.

The development of a hybrid in a plant breeding program involves three steps: (1) the selection of plants from various germplasm pools for initial breeding crosses; (2) the selfing of the selected plants from the breeding crosses for several generations to produce a series of inbred lines, which, while different from each other, breed true and are highly homozygous and (3) crossing the selected inbred lines with different inbred lines to produce the hybrids. During the inbreeding process, the vigor of the lines decreases. Vigor is restored when two different inbred lines are crossed to produce the hybrid. An important consequence of the homozygosity and homogeneity of the inbred lines is that the hybrid created by crossing a defined pair of inbreds will always be the same. Once the inbreds that give a superior hybrid have been identified, the hybrid seed can be reproduced indefinitely as long as the homogeneity of the inbred parents is maintained.

Plants of the present disclosure may be used to produce, e.g., a single cross hybrid, a three-way hybrid or a double cross hybrid. A single cross hybrid is produced when two inbred lines are crossed to produce the F1 progeny. A double cross hybrid is produced from four inbred lines crossed in pairs (A×B and C×D) and then the two F1 hybrids are crossed again (A×B) times (C×D). A three-way cross hybrid is produced from three inbred lines where two of the inbred lines are crossed (A×B) and then the resulting F1 hybrid is crossed with the third inbred (A×B)×C. Much of the hybrid vigor and uniformity exhibited by F1 hybrids is lost in the next generation (F2). Consequently, seed produced by hybrids is consumed rather than planted.

This invention can be better understood by reference to the following non-limiting examples. It will be appreciated by those skilled in the art that other embodiments of the invention may be practiced without departing from the spirit and the scope of the invention as herein disclosed and claimed.

### EXAMPLES

### Example 1

Genetically diverse soybean accessions were grown under prolonged exposure to dicamba under field conditions to identify significant marker-trait associations.

### Materials and Methods

### Plant Materials and Data Collection

A total of 382 genetically diverse soybean accessions with maturity groups (MG) ranging from MG 3 to 5 were used in this study. These comprise a subset of the USDA Soybean Germplasm Collection and originated from 15 countries, including Algeria (2), China (226), Costa Rica (1), Georgia (2), Indonesia (1), Japan (38), Nepal (1), North Korea (20), Russia (5), South Africa (1), South Korea (32), Taiwan (3), United States (40), and Vietnam (5). Five accessions have unknown origins. The USDA Soybean Germplasm Collection was genotyped with the SoySNP50K BeadChip. SNPs were converted to numerical format (0, 1, and 2 for the homozygous minor allele, heterozygous, and homozygous major allele, respectively), and were excluded based on minor allele frequency (MAF)<0.05 resulting in 31,957 SNPs. The across-genome SNP density on a chromosome basis was 1,598, ranging from 1,186 (Chr. 11) to 2,619 (Chr. 18).

Field trials were conducted in three environments for two years (2020-2021) using a two-replicate randomized complete block design in Portageville, MO (36°23′44.2″N lat; 89°36′52.3″W long) and Clarkton, MO (36°29′14.8″N lat; 89°57′39.0″W long). Each plot consisted of a single 2.13 m long row spaced 0.76 m apart. Both farm locations have been exposed to prolonged and homogeneously distributed off-target dicamba damage since 2017, where significant yield losses due to off-target dicamba exposure have been reported between non-DT and DT soybean genotypes.

Each year, genotypes were visually assessed for off-target dicamba damage once in the early reproductive stage between R1 to R3 (approximately 100 to 130 DAP). Lines were rated on a 1 to 5 scale with 0.5 increments. In summary, a rating of 1 showed none to minimal visual dicamba damage symptoms, including the typical crinkling and cupping of the newly-developing leaves, reduced canopy coverage, and plant stunting; a rating of 2 showed moderate tolerance with limited cupping of the newly-developing leaves and no visual impact on canopy coverage and vegetative growth; a 3 rating showed accentuated cupping of the newly-developing leaves and moderate reduction in canopy area and vegetative growth; a 4 rating showed severe cupping of the newly-developing leaves and pronounced reduction in canopy area and vegetative growth, and a rating of 5 showed extreme dicamba damage symptoms including severe cupping of the newly-developing leaves and intense reduction in canopy coverage and vegetative growth.

Adjusted means across environments were calculated using the function ‘ls_means’ of the R package ‘lmerTest’ (available at https://www.r-project.org/) based on a mixed-effects linear model conducted with the package ‘lme4’. The model included the fixed effect of genotype, the random interaction between genotype and environment (G×E), the random effect of environment, and the nested random effect of replication within the environment. To measure the consistency and inter-relatedness of the damage ratings across environments, Cronbach's alpha (α) score was calculated each year using the R package ‘psych’ (Revelle, 2021) based on Eq. 1.

$$α = \frac{k \times \overline{c}}{\overline{v} + ( k - 1 ) \overline{c}} \qquad Eq . 1$$

Where k represents the number of observations of off-target dicamba damage; c̅ is the average inter-item covariance of off-target dicamba damage scores between each pair of environments averaged for all pairs of environments; and v̅ is the average variance of off-target dicamba damage scores across all environments.

### Genome-Wide Association Study

Two models have been implemented to detect significant marker-trait associations. The Bayesian-information and Linkage-disequilibrium Iteratively Nested Keyway (BLINK) model was conducted using the adjusted means across environments as phenotypic input in the R package ‘GAPIT’. It is an enhanced methodology based on the Fixed and Random Model Circulating Probability Unification (FarmCPU). In summary, FarmCPU conducts two fixed-effect models iteratively and a filtering process to select a set of pseudo-SNPs that are not in linkage disequilibrium with each other as covariates. The first model tests one SNP at a time with multiple associated markers fitted as covariates to account for population stratification. The main goal is to control false positives and reduce false negatives, as well as calculate the p-values for all testing SNPs. The second model selects the covariate markers to directly control false associations instead of kinship. BLINK eliminates the requirement that genes underlying a trait are equally distributed across the genome, and also replaces the Restricted Maximum Likelihood (REML) with Bayesian Information Content (BIC) in a fixed-effect model to boost computing speed.

To account for the variable patterns of genotype responses to off-target dicamba in different environments, a model that allows the inclusion of the population structure in interaction with environments was considered. The model was fitted with ASREML-R (VSN-International, England). Considering that y_ijk represents the kth (k=1, 2) response of the ith (i=1, 2, . . . , 382) genotype in the jth (j=1, . . . , 3) environment, the GWAS was conducted using the following linear mixed model in matrix form:

y=E+R:E+PC_{1:10}+E×PC₁+E×PC₂+ . . . +E×PC₁₀+xₖ+L+e Eq. 2

Where E corresponds to the main effect of the environments; R:E represents the effect of the replicates nested within environments; PC_{1:10} are the first 10 principal components (PC) derived from decomposing the G matrix using a principal component analysis (PCA) and are included in the model for correcting for population structure; the interactions between the first 10 components PC_{1:10} and environments were also included with the E×PC₁ term; the xₖ term corresponds to the k^{th} molecular marker associated with βₖ (marker effect). All of the previous model terms were considered fixed terms. The random effect L is associated with the main effect of the genotypes, and e corresponds to the error term which captures the unexplained variability.

To control the comparison-wise error rate, the effective number of independent tests (M_{eff}) was derived by considering the eigenvalue decomposition of the matrix of correlations between markers. The resulting test was adjusted using the M_{eff} with the following correction:

αₚ=1−(1−αₑ)^{-M}^{_{eff}}

Where αₚ is the comparison-wise error rate and αₑ corresponds to the experiment error-wise (αₑ=0.05).

### Results

### Phenotypic Distribution

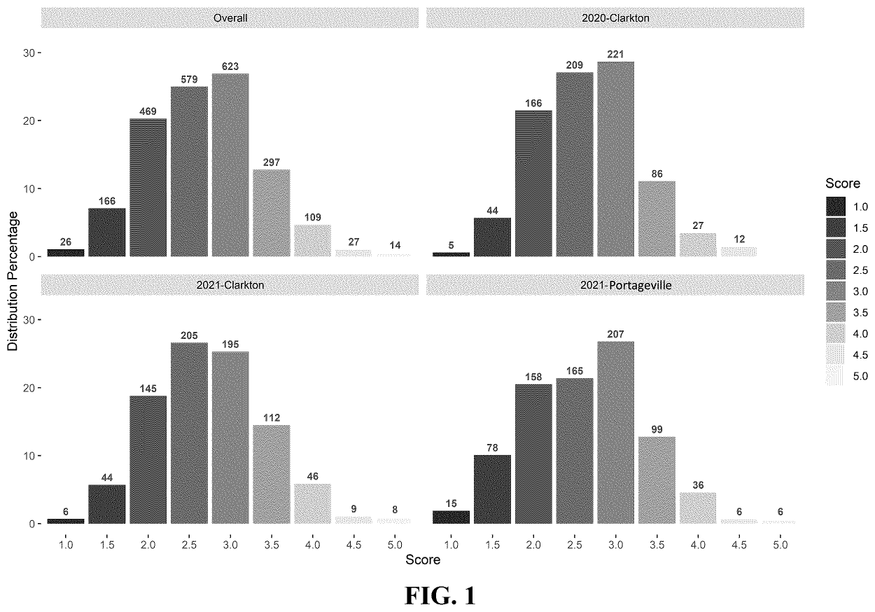

Across the three testing environments, the frequency of off-target dicamba damage scores was consistent and normally distributed with over 45% of the observations between scores of 2 and 3 (moderately tolerant) and 39% between 3 and 3.5 (moderately susceptible) (FIG. 1). Roughly 8% of the observations were either under the score of 2 (highly tolerant) or above the score of 4 (highly susceptible).

Across environments, scores were consistent with an overall Cronbach's alpha (α) score of 0.89 (C.I 0.87 to 0.91). Within environments, scores ranged from 0.85 to 0.89, indicating minor error variance and discrepancy across replications (Table 1). Cronbach's α can be interpreted as the correlation of the test with itself, of which the error variance can be obtained by subtracting the squared α from 1.00. The error variance ranged from 0.21 to 0.26 which indicates consistency and inter-relatedness of the damage scores across and within environments. Scores above 0.70 (error variance of 0.51) are often considered acceptable. In plant breeding, reliability refers to multiple measurements across different environments that are independent of each other and may be explored as a new measurement of the influence of genetic versus nongenetic effects as opposed to heritability.

TABLE 1
Summary of Cronbach's alpha (α) across testing environments.

| Environment | Alpha (α)¹ | C.I (95%)² | | Error³ | Correlation⁴ | Mean⁵ | S.D⁶ |
|---|---|---|---|---|---|---|---|
| Overall | 0.89 | 0.87 | 0.91 | 0.21 | 0.62 | 2.70 | 0.59 |
| 2020-Clarkton | 0.86 | 0.84 | 0.88 | 0.26 | 0.61 | 2.70 | 0.66 |
| 2021-Clarkton | 0.86 | 0.85 | 0.87 | 0.26 | 0.61 | 2.80 | 0.72 |
| 2021-LeeFarm | 0.89 | 0.87 | 0.91 | 0.21 | 0.64 | 2.60 | 0.69 |

¹Standardized alpha (α) based upon the correlations.
²Confidence interval (95%) of standardized alpha (α) score.
³Estimated error variance was obtained by subtracting the squared α from 1.00.
⁴Inter-item average Pearson's correlation.
⁵Average of off-target dicamba damage scores in each environment.
⁶Standard deviation of the observed scores in each year.

### Significant Marker-Trait Associations

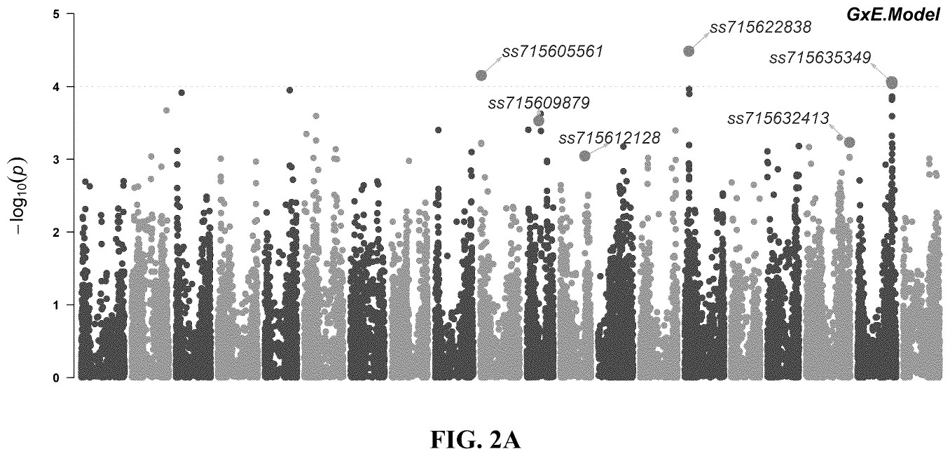

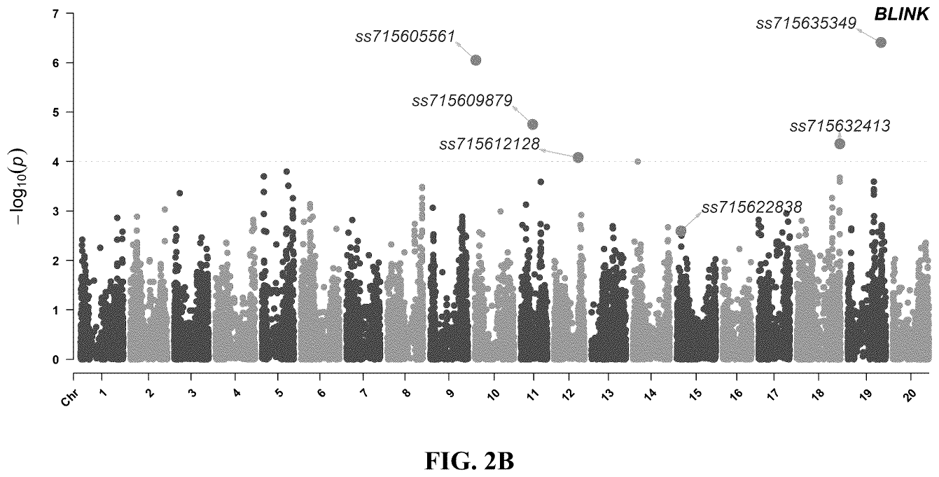

The calculated effective number of independent tests (M_{eff}) was 575, which returned a threshold of marker-trait association significance of logarithm of odds (LOD) of approximately 4.0. Using the proposed model that allows the inclusion of the population structure in interaction with environments (Eq. 2), three significant marker-trait associations were detected in chromosomes 10 (LG O), 15 (LG E), and 19 (LG L) (FIG. 2A). The SNP ss715622838 located at 5,457,236 bp of chromosome 15 (Genome assembly version Wm82.a2) had the highest LOD (4.5) with a favorable allele frequency of 13.9%. This SNP is located within the gene Glyma15g07710 which encodes a copper-containing oxidoreductase enzyme, tyrosinase. These copper containing enzymes can oxidize a wide range of aromatic compounds, including the oxidation of o-diphenols to their corresponding o-quinones. Phase I of herbicide detoxification in plants involves oxidation by cytochrome P450s or hydrolysis by carboxylesterases. Given the structural similarities between dicamba and o-diphenols, tyrosinase may also be involved in the hydroxylation of dicamba in soybean. The SNP ss715605561 located at 1,227,933 bp of chromosome 10 (Genome assembly version Wm82.a2) had the second-highest LOD (4.2) with a favorable allele frequency of 8.0%. It is located within the gene Glyma10g01700 which encodes a multidrug resistance protein (MRP). MRPs are essential in phase III of plant herbicide detoxification by facilitating the transport of glucose- or glutathione-herbicide conjugates into the vacuole. Lastly, the SNP ss715635349 located at 44,580,800 bp of chromosome 19 (Genome assembly version Wm82.a2) had a LOD of 4.1 with a favorable allele frequency of 28.3% (Table 2). Interestingly, ss715635349 is located within the gene Glyma19g37108, a uridine diphosphate (UDP)-dependent glycosyltransferase gene. This genomic region contains additional five UDP-glycosyltransferase genes. The conjugation of Phase I-hydroxylated herbicides to endogenous sugar molecules such as glucose is catalyzed by UDP-dependent glycosyltransferases and represents an important phase II reaction of plant herbicide detoxification.

Four significant marker-trait associations were detected in chromosomes 10 (LG O), 11 (LG B1), 18 (LG G), and 19 (LG L) using the BLINK model (FIG. 2B). The SNP ss715635349 located at 44,580,800 bp of chromosome 19 had the highest LOD (6.4) followed by the SNP ss715605561 located at 1,227,933 bp of chromosome 10 (LOD of 6.1). The SNP ss715609879 had a LOD of 4.8 with a favorable allele frequency of 24.0%. It is positioned at 15,740,804 bp of chromosome 11 (Genome assembly version Wm82.a2) and is located within Glyma11g29391, a lipid phosphate phosphatase gene. Lastly, the SNP ss715632413 located at 57,025,570 bp of chromosome 18 (Genome assembly version Wm82.a2) had a LOD of 4.4 with a favorable allele frequency of 19.4% (Table 2). Two genes with detoxification-related annotations (Glyma18g291800 and Glyma18g291700) are located within 50-kb of ss715632413.

TABLE 2
Summary of significant marker-trait associations
identified using the G × E model and BLINK.

| | | | | | LOD³ | |
|---|---|---|---|---|---|---|
| | | | Position | MAF | G × E | |
| SNP | Chromosome | | (bp)¹ | (%)² | Model | BLINK |
| ss715635349 | 19 | (LG L) | 44,580,800 | 28.3 | 4.1 | 6.4 |
| ss715605561 | 10 | (LG O) | 1,227,933 | 8.0 | 4.2 | 6.1 |
| ss715609879 | 11 | (LG B1) | 15,740,804 | 24.0 | 3.5 | 4.8 |
| ss715632413 | 18 | (LG G) | 57,025,570 | 19.4 | 3.2 | 4.4 |
| ss715622838 | 15 | (LG E) | 5,457,236 | 13.9 | 4.5 | 2.6 |
| ss715619759 | 14 | (LG B2) | 6,460,927 | 19.9 | 1.7 | 4.0 |
| ss715590768 | 5 | (LG A1) | 32,594,828 | 9.0 | 3.9 | 3.8 |
| ss715592527 | 5 | (LG A1) | 2,516,484 | 3.3 | 2.3 | 3.7 |
| ss715632432 | 18 | (LG G) | 57,206,151 | 43.6 | 3.0 | 3.7 |
| ss715632412 | 18 | (LG G) | 57,013,050 | 26.7 | 3.0 | 3.6 |

¹Position in the genome reported as base pairs (Genome assembly version Wm82.a2).
²Minor allele frequency reported in percentage.
³LOD, the logarithm of odds calculated as the negative logarithm of the observed p-value for each model. The G × E model is described in Eq. 2 and BLINK is described in Huang et al. (2019).

### Marker Effect on Observed Phenotype

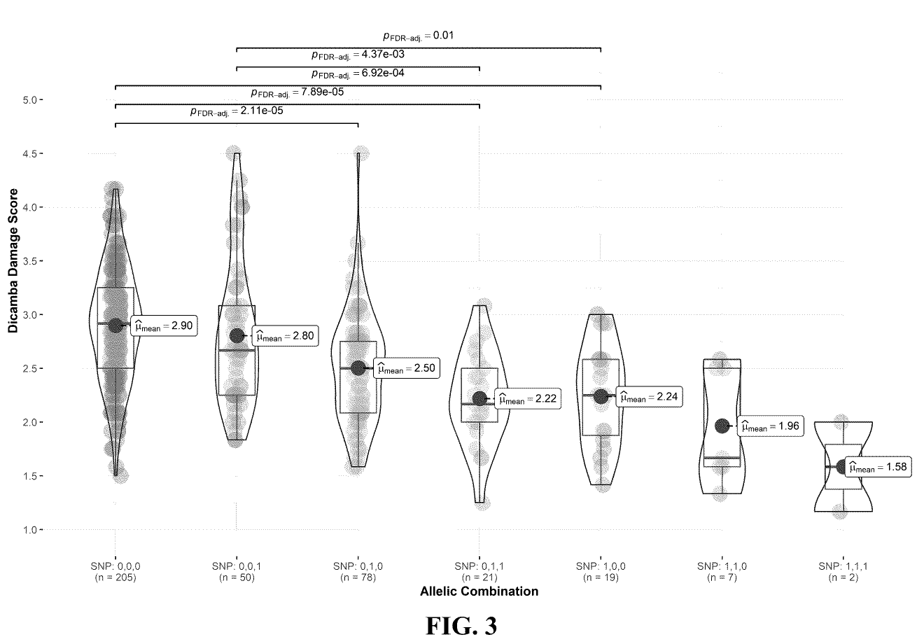

To assess the effect of significant SNPs on the observed phenotype, genotypes were classified according to the allelic combination of the significant SNPs ss715605561 (Chr. 10), ss715635349 (Chr. 19), and ss715632413 (Chr. 18). Favorable alleles were represented as 1 whereas unfavorable alleles were represented as 0. For instance, SNP: 0,0,0 represents the allelic combination where ss715605561, ss715635349, and ss715632413 are unfavorable, and SNP: 1,1,1 represents all favorable alleles. The mean score of dicamba damage in genotypes carrying all three favorable alleles was 1.58, whereas the mean score of genotypes carrying all three unfavorable alleles was 2.90 (FIG. 3). The presence of the favorable allele of ss715605561 (SNP: 1,0,0; SNP: 1,1,0, and SNP: 1,1,1) and ss715635349 (SNP: 0,1,0; SNP: 0,1,1; SNP: 1,1,1) significantly reduced the overall damage from off-target dicamba as compared to all non-favorable alleles, while genotypes carrying only the favorable allele for ss715632413 (SNP: 0,0,1) did not show significant differences to the genotypes carrying only unfavorable alleles (FIG. 3).

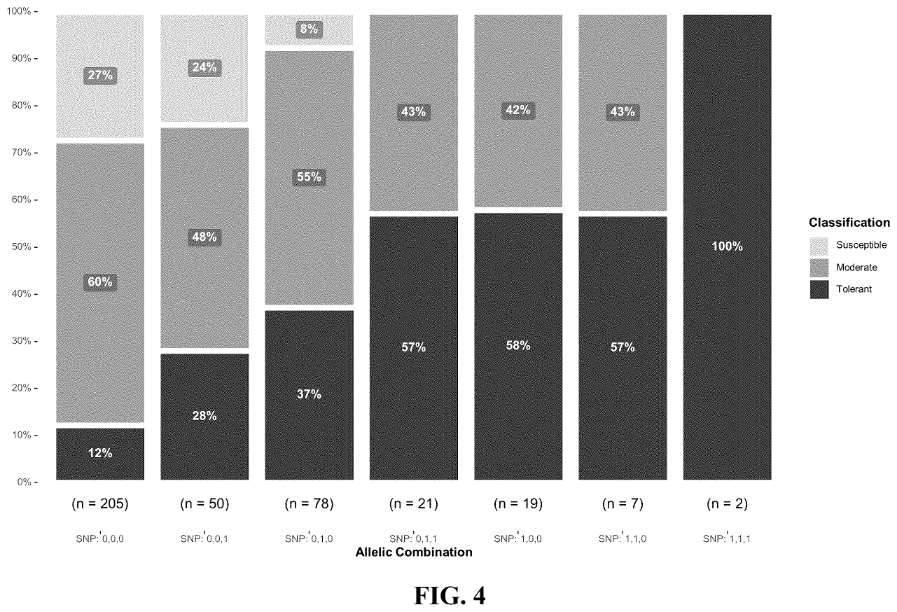

In addition, to assess the potential of differentiating response classes using the significant SNPs, genotypes were classified as tolerant (score<=2.5), moderate (2.5<score=<3.5), and susceptible (score>3.5). The classification distribution based on the allelic combination of these SNPs showed a substantial reduction in the susceptible class with the inclusion of the favorable alleles of ss715605561 and ss715635349 (FIG. 4). On the other hand, the combination SNP: 0,0,0 had the highest concentration of susceptible (27%) and moderate (60%) genotypes and the lowest concentration of tolerant genotypes (12%) (FIG. 4). Interestingly, no susceptible genotypes were observed in combinations SNP: 0,1,1, SNP: 1,0,0, SNP: 1,1,0, and SNP: 1,1,1, indicating that the selected SNPs can accurately select genotypes with higher tolerance response to off-target dicamba exposure.

### Discussion

Soybean tolerant to postemergence applications of dicamba was developed under the premise of overcoming weeds resistant to glyphosate as well as allowing rotation and/or mixtures of herbicides to preserve biotechnology-based weed management strategies and maximize its efficacy. The insertion of the bacterial gene dicamba monooxygenase (DMO) from Pseudomonas maltophilia (Strain DI-6) encoding the enzyme dicamba O-demethylase allows DT plants to metabolize dicamba to 3,6-dichlorosalicylic acid (DCSA), inactivating its herbicidal activity before it accumulates to toxic levels when expressed from either the nuclear genome or chloroplast genome of genetically engineered plants. In 2016, the first commercial dicamba-tolerant soybean cultivar was released in the United States and rapidly took over nearly 55% of the soybean acreage. As the incidents of off-target damage widely spread across soybean-growing states, many reports in the literature investigated the relationship between damage and potential yield losses. Soybean is two to six times more sensitive to dicamba when exposed at the early reproductive stage as compared to the vegetative stage. Interestingly, certain genetic backgrounds consistently show natural tolerance to off-target dicamba exposure with minimal symptoms and yield losses.

In this study, a total of 382 genetically diverse soybean accessions ranging from MG 3 to 5 were phenotypically screened based on the severity of damage across three environments subjected to prolonged off-target dicamba exposure. Most accessions showed a moderate response, either moderately tolerant or moderately susceptible, with approximately 8% showing high tolerance (scores<2) and susceptibility (scores>4). No differences in off-target dicamba damage were observed across MG, MG 3: average damage of 2.8, MG 4: average damage of 2.7, MG 5: average damage of 2.5). Late-maturing soybean genotypes are associated with lower off-target dicamba damage due to a longer window to detoxify from low rates of dicamba between planting and flowering compared with early-maturing genotypes. Tolerant soybean accessions were identified across all MG, confirming that natural tolerance to off-target dicamba may be caused by physiological mechanisms other than the length of time for recovery. In addition, no substantial geographical effects have been identified across continents, although accessions derived from Asia (average damage of 2.6) had on average lower off-target dicamba damage as compared to accessions derived from the Americas (average damage of 3.0). Specifically, South Korea (average damage of 2.5), Japan (average damage of 2.6), and China (average damage of 2.7) had on average lower off-target dicamba damage as compared to accessions derived from the United States (average damage of 2.3) and Costa Rica (average damage of 3.1), although the number of accessions was highly unbalanced across countries.

Plant introduction (PI) 424005 (average damage of 1.2, South Korea), PI 424038-B (average damage of 1.3, South Korea), PI 561701 (G88-20092, average damage of 1.4, United States), PI 603497 (average damage of 1.4, China), and PI 342434 (average damage of 1.5, Japan) were the five most tolerant accessions. On the other hand, PI 547862 (L83-570, average damage of 4.1, United States), PI 552538 (Dunbar, average damage of 4.2, United States), PI 598124 (Maverick, average damage of 4.3, United States), PI 603675 (average damage of 4.5, China), and PI 597387 (Pana, average damage of 4.5, United States) were the five most susceptible. Interestingly, four out of the five most susceptible accessions are genetically related to cultivars that widely contributed to the genetic basis of modern soybean cultivars in the United States. For instance, Maverick and Pana are derived from LN86-4668, which is a progeny of Fayette (PI 518674, direct progeny of PI 88788). PI 88788, which has been widely used as a genetic source of resistance to soybean cyst nematode (Heterodera glycine Ichinohe), is susceptible to off-target dicamba (average damage of 3.5). In general, soybean [Glycine max (L.) Merr.] shows a moderate response to off-target dicamba, and yield losses are expected when prolonged exposure occurs. Furthermore, genetic variation conferring higher tolerance to off-target dicamba appears to be rare in landraces, highlighting the value of the USDA Soybean Germplasm Collection to restore economic-important alleles lost during domestication and intensive breeding. This has been the case in multiple economically important traits, including resistance to soybean cyst nematode, root-knot nematodes (Meloidogyne spp.), foliar feeding insects, and brow stem rot (Phialophora gregata).

Two models were used to identify marker-trait associations regulating the response of soybean to off-target dicamba. BLINK minimizes false-positive associations and greatly improve computational efficiency in larger datasets. To account for possible underlying population structures affecting the observed response to off-target dicamba in different environments, a model including the first 10 principal components (PC) derived from decomposing the G matrix as well as the interaction between each PC and the environment was developed. In addition, this model allows the inclusion of all observed phenotypes (three environments×two replications per genotype) as opposed to only one adjusted mean per genotype, substantially reducing the ‘Curse of Dimensionality’ where the number of independent variables is far higher than the number of samples that are often seen in genomic studies. We observed that both models identified significant associations between ss715635349 (Chr. 19) and ss715605561 (Chr. 10) and the response to off-target dicamba. The BLINK model identified additional significant marker-trait associations on Chrs. 11 (ss715609879), 14 (ss715619759), and 18 (ss715632413), while the G×E Model identified an additional significant marker-trait association on Chr. 15 (ss715622838). The significant SNPs identified by both models are located within candidate genes with annotated functions involved with different phases of herbicide detoxification in herbicides.

Phase I typically involves oxidation by cytochrome P450s or hydrolysis by carboxylesterases. These reactions introduce a reactive functional group suitable for subsequent metabolism and detoxification since this initial oxidation step may not lead to complete detoxification. Phase II detoxification reactions involve the conjugation of herbicides with reduced glutathione or glucose and are catalyzed by glutathione S-transferases or UDP-dependent glycosyltransferases. The SNP ss715635349 is located within the gene Glyma19g37108, a uridine diphosphate (UDP)-dependent glycosyltransferase gene. Within a 30-kb window from ss715635349 (44,550,000 to 44,610,000 bp) there are another five UDP-dependent glycosyltransferases genes. The frequency of the favorable allele of ss715635349 suggests that the ability to complete phase II detoxification of dicamba is relatively common (28.3%) and may explain the overall moderate response of soybean to off-target dicamba. Phase III of herbicide detoxification involves the active transport (ATP-dependent) of non-phytotoxic herbicide conjugates into the vacuole by proteins in the multidrug resistance-associated protein (MRP) family or by other transport mechanisms. The SNP ss715605561 is located within the MRP gene Glyma10g01700. The low favorable allele frequency (8.0%) could explain the rare occurrence of highly dicamba-tolerant soybean phenotypes. Based on these results, without being limited by theory, it is believed that most soybean genotypes conduct phase I ring hydroxylation and phase II detoxification of dicamba with glucose but have a rate-limiting step in the final phase III transport of non-phytotoxic dicamba-glucose conjugates into the vacuole.

Although the response to off-target dicamba appears to be a highly complex trait regulated by multiple genes involved in several biochemical pathways, the combination of ss715605561 (Chr. 10), ss715635349 (Chr. 19), and ss715632413 (Chr. 18) can accurately distinguish among tolerant to susceptible genotypes. Accessions carrying the favorable alleles for these SNPs showed the lowest average off-target dicamba damage and the highest frequency of tolerant and moderate classes. Future plant breeding research will utilize and apply these alleles in marker-assisted selection programs targeting identification and development of genotypes with higher tolerance to off-target dicamba. Additionally, molecular physiology research is currently underway to investigate expression patterns and functional roles of the alleles and encoded proteins identified by our GWAS analysis.

### Conclusions

Widespread adoption of DT production systems has frequently resulted in yield losses in non-DT soybean genotypes from off-target dicamba exposure. Identification of genetic sources of tolerance and genomic regions conferring higher tolerance to off-target dicamba in non-DT soybean genotypes may sustain and improve other non-DT soybean production systems, including the growing niche markets of organic and conventional soybean. Herein, we report several genetically diverse accessions that can be used as genetic sources for improved tolerance to off-target dicamba. Two genomic regions on Chrs. 10 and 19 were identified that may be directly associated with the ability of soybean to detoxify dicamba and/or transport non-phytotoxic dicamba metabolites into the vacuole. Three significant SNPs accurately distinguished between tolerant and susceptible genotypes. With current plant breeding techniques and the advancements in targeted gene-editing techniques, these results may facilitate identifying and developing conventional soybean cultivars with improved tolerance to off-target dicamba as well as other synthetic auxin herbicides.

### Example 2

A further genome-wide association study (GWAS) was conducted to identify novel marker-trait associations and expand on previously identified genomic regions in a new population with different genetic backgrounds. A machine learning (ML)-GWAS pipeline incorporating a supervised feature dimension reduction based on Variable Importance in Projection (VIP) and classification algorithms was implemented to identify the combination of SNPs that provided the highest classification accuracy for off-target dicamba response. Identification and characterization of the genetic architecture of soybean tolerance to off-target dicamba and the development of non-DT tolerant genotypes may sustain the production and adoption of other genetically engineered herbicide-tolerant soybean production systems in regions severely affected by off-target dicamba exposure, as well as the expanding niche markets based of organic and conventional soybean.

### Materials and Methods

### Plant Material and Genomic Data

Soybean genotypes consisted of 551 non-DT advanced breeding lines derived from 232 unique bi-parental populations. In addition, 18 commercial cultivars [14 DT and four non-DT glyphosate [(N-(phosphonomethyl)glycine)]-tolerant (GT)] were included in the study as controls to confirm the presence of off-target dicamba exposure and assess the homogeneity of off-target dicamba distribution. In 2019, plant materials consisted of 210 advanced breeding lines, three GT commercial cultivars, and seven DT commercial cultivars. In 2020, plant materials consisted of 204 advanced breeding lines, three GT commercial cultivars, and six DT commercial cultivars. In 2021, 209 advanced breeding lines, three GT commercial cultivars, and 11 DT commercial cultivars were evaluated. In the study, some overlapping of genotypes across years was observed, hence the total number of genotypes evaluated across environments included more than 551 advanced breeding lines and 18 commercial cultivars. The maturity group (MG) of genotypes ranged from 4-early to mid-5. MG was noted as the number of days after August 1^{st} when 95% of pods on the main stem had reached mature brown color. Relative maturity (RM) was calculated as days earlier or later than reference controls and was used to assign MG, where 4-early=4.0 to 4.3 (88 genotypes), mid-4=4.4 to 4.6 (127 genotypes), 4-late=4.7 to 4.9 (171 genotypes), 5-early=5.0 to 5.3 (138 genotypes), and mid-5=5.4 to 5.6 (27 genotypes). All soybean breeding lines were genotyped using the Illumina Infinium BARCSoySNP6K BeadChip at the USDA-ARS Soybean Genomics and Improvement Laboratory (Beltsville, MD). A total of 4,970 SNPs were obtained after filtering based on minor allele frequency (MAF)<0.05.

### Field Experiments and Data Collection

Nine environments (combination of location, field, and year) under prolonged off-target dicamba exposure were used to conduct field experiments for three years (2019-2021) in Portageville, MO. Genotypes were distributed in field trials based on MG. Each field trial was arranged in a three-replicate randomized complete block design where each plot consisted of four 3.66 m long rows spaced 0.76 m apart. The homogeneity of off-target dicamba exposure was assessed and confirmed using an inhomogeneous Poisson marked point process based on the spatial distribution of the relative yield performance between GT and nearby DT commercial cultivars.

Soybean genotypes were visually assessed for off-target dicamba damage on a 1 to 4 scale with 0.5 increments between R1 and R3, as described in Example 1. The consistency and reliability of scores across and within environments were confirmed using Pearson's correlation coefficients and Cronbach's alpha, respectively.

Damage ratings were adjusted across environments as in Example 1. Genotypes were classified into three categories based on the adjusted off-target dicamba damage: tolerant when damage scores≤2, moderate>2, ≤3, and susceptible>3.

### Genome-Wide Association Study

Two linear regression-based models were utilized to conduct GWAS, including the Fixed and Random Model Circulating Probability Unification (FarmCPU) and the Bayesian-information and Linkage-disequilibrium Iteratively Nested Keyway (BLINK). In addition, one ML-GWAS pipeline incorporating feature dimension reduction and classification algorithms was implemented. In summary, FarmCPU maximizes the advantages of mixed linear models and stepwise regression by using them iteratively. It substitutes kinship with a set of molecular markers fitted as fixed effects that are tested one at a time across the genome. The molecular markers are optimized in a restricted maximum likelihood method in a mixed linear model with variance and covariance defined by the set of pre-selected molecular markers, reducing the risk of model overfitting. BLINK is an improved version of FarmCPU that discards the assumption that genes associated with a trait are evenly spread across the genome. It replaces the restricted maximum likelihood method with Bayesian Information Content (BIC) to improve computing speed. Both FarmCPU and BLINK models were conducted using the R (R Core Team, 2023) package “GAPIT”.

### Feature Selection and Machine Learning Classification Algorithms

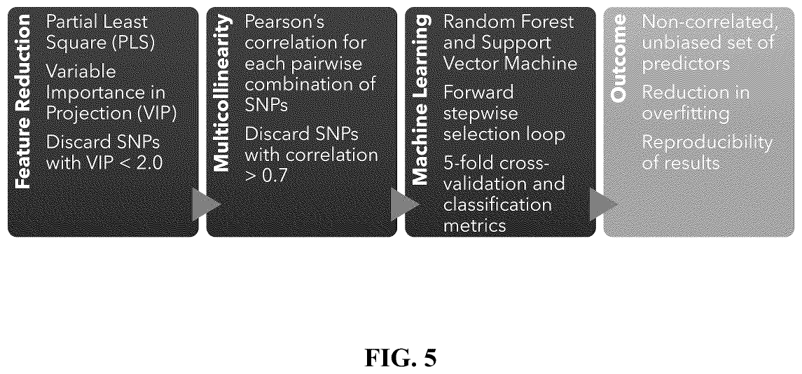

The ML-GWAS pipeline to identify the combination of predictors yielding the highest prediction accuracy was implemented following the protocol described in FIG. 5. A Partial Least Square (PLS) model was fitted using the off-target dicamba damage scores as the variable response and the 4,970 SNPs as predictors. The components' coefficients were trained using a 10-fold cross-validation to achieve a low validation error. The relative importance of each predictor in the components was represented by the Variable Importance in Projection (VIP) scores. The analysis was conducted in R (R Core Team, 2023) using the package ‘pls’ to fit the PLS model and ‘plsVarSel’ to obtain the VIP scores.

The SNPs with VIP scores<2.0 were discarded. Among the SNPs with VIP≥2.0, Pearson's correlation coefficients were calculated for each possible pairwise combination. For each iteration, if the pair-wise correlation was <0.7, both SNPs were kept. The SNP with the lowest VIP was discarded when an absolute pairwise correlation≥0.7 occurred. The loop finished after the last possible pair-wise correlation was calculated. The objective of this filtering step was to limit overfitting and multicollinearity by discarding highly correlated predictors with low relative importance to the response variable.

The non-correlated selected SNPs with VIP≥2.0 were included as predictors in the Random Forest (RF) and Support Vector Machine (SVM) models in a forward stepwise selection loop to identify the combination of SNPs yielding the highest classification accuracy. The selection loop started by fitting the SNP with the highest VIP, followed by adding each SNP one at a time. The SNP yielding the highest accuracy in the preceding iteration was retained in the subsequent loop, of which the classification accuracy was calculated with an additional SNP. The loop concluded when no further improvement in the classification accuracy was observed by the addition of another SNP, thereby identifying the optimal combination of predictors. To evaluate the impact of overfitting on the prediction accuracy of both models, the loop continued despite no additional gain in classification accuracy, and classification accuracy metrics were recorded for each iteration.

Each iteration was analyzed with 5-fold cross-validation and classification accuracy metrics were recorded. The overall accuracy of each iteration was computed using eq. 1. Class accuracy is represented by the proportion of true positives (TP) and true negatives (TN) for individual classes (sum of TP, TN, false positive (FP), and false negative (FN)) (Eq. 2). Precision is described as the number of TP by the number of predicted positives (TP+FP) for individual classes (Eq. 3). Specificity is defined by the ratio of TN by TN and FP for individual classes (Eq. 4).

$$Overall Accuracy = \frac{No . of Correct Classifications}{Total No . of Samples} \times 100 \% \qquad 1 )$$

$$Class Accuracy = \frac{TP + TN}{TP + TN + FP + FN} \qquad 2 )$$

$$Precision = \frac{TP}{TP + FP} \qquad 3 )$$

$$Specificity = \frac{TN}{TN + FP} \qquad 4 )$$

where, TP=True Positive (correctly predicted as the positive class); TN=True Negative (correctly predicted as the negative class); FP=False Positive (incorrectly predicted as the positive class); FN=False Negative (incorrectly predicted as the negative class).

RF and SVM machine learning models were used for the multi-class prediction problem. These were chosen based on their efficacy in handling data in which the number of predictors is larger than the number of observed samples, as well as a providing satisfactory balance between the variance-bias trade-off. RF is a supervised learning algorithm based on the assembly of multiple decision trees. It conducts feature selection and generates non-correlated decision trees making it feasible to include a high number of predictors in the model. The SVM model places flexible hyperplanes among classes, being particularly useful in classification problems. The model provides flexibility to identify combinations of adjustable parameters that optimize model performance while mitigating the risk of overfitting.

The RF model was conducted using the R package ‘randomForest’ with the square root of p predictors (number of predictors) randomly selected at each split. The SVM model was conducted using the R package ‘e1071’ with the kernel defined as ‘radial’. The optimal combination of trainable parameters was provided using the function ‘tune’. The final model was tuned using a grid search for the cost ranging from 0.01, 0.1, 1, 10, 100, and 1000, and gamma ranging from 0.0001, 0.001, 0.01, 0.5, and 1.

### Results

### Phenotypic Distribution

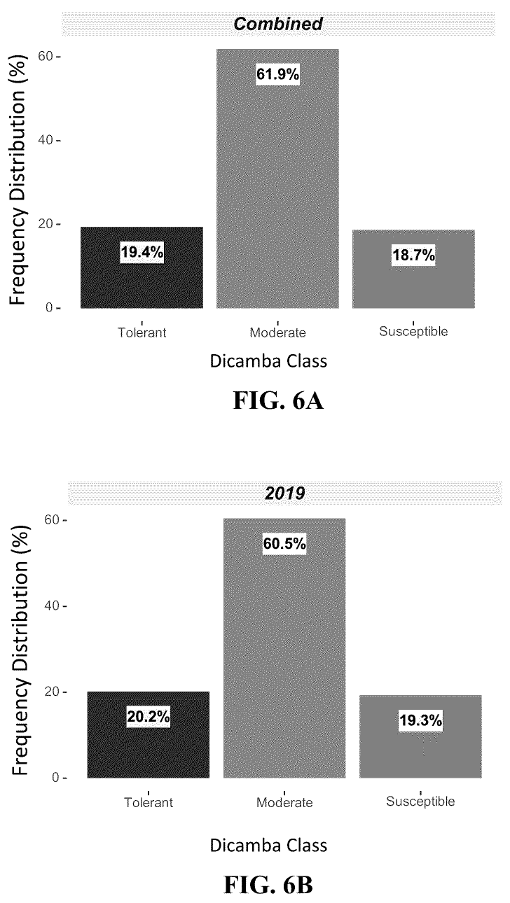

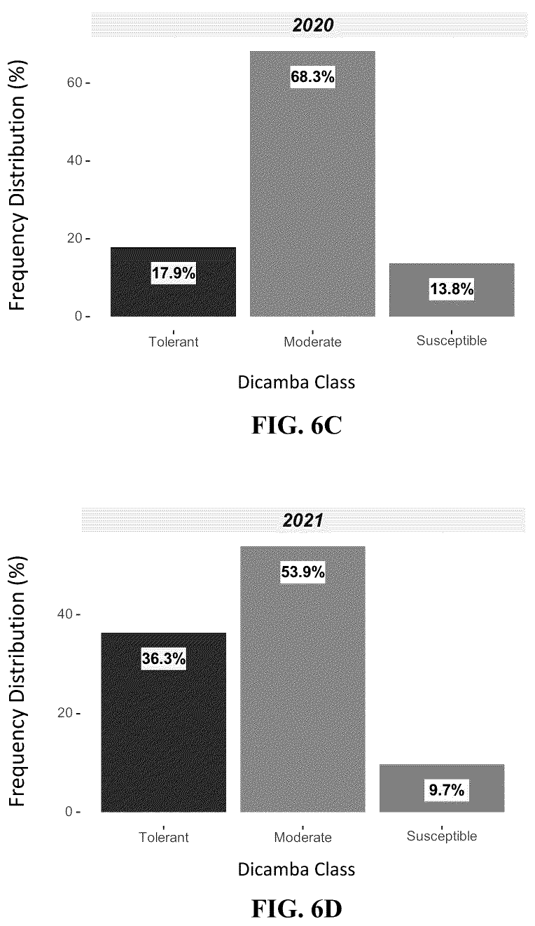

Across all testing years (2019-2021), a total of 107 genotypes were classified as tolerant (19.4%), 341 as moderate (61.9%), and 103 as susceptible (18.7%) (FIGS. 6A-D). The distribution was relatively uniform across the years, although the frequency of susceptible genotypes declined over the years as a result of the potential indirect selection of tolerant genotypes based on favorable agronomic traits and yield in environments exposed to prolonged off-target dicamba. Indirect selection has been documented in soybean for multiple traits, including off-target dicamba tolerance, adaptation and maturity, seed size, and grain yield.

### GWAS Results

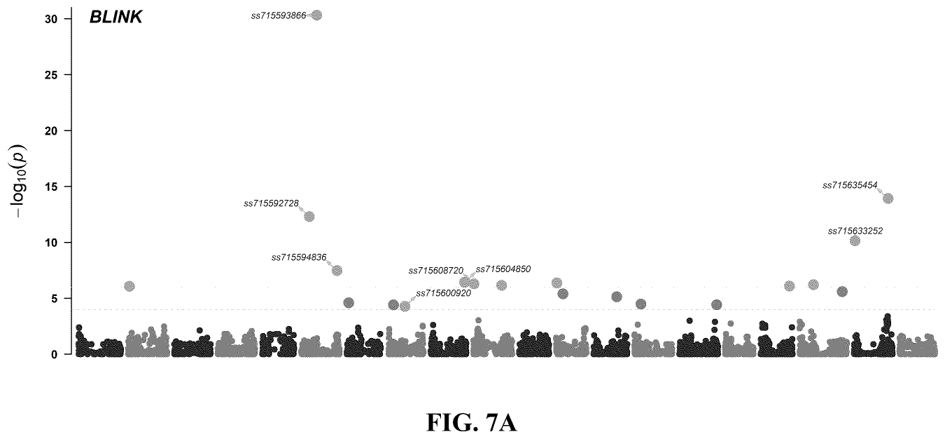

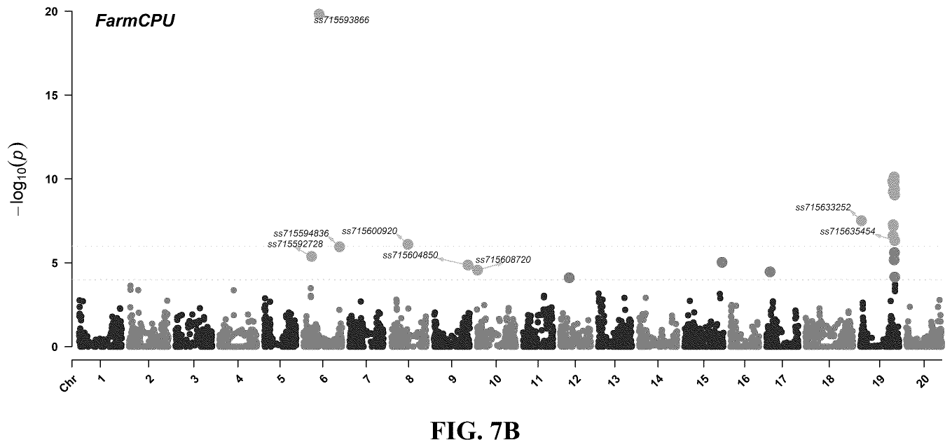

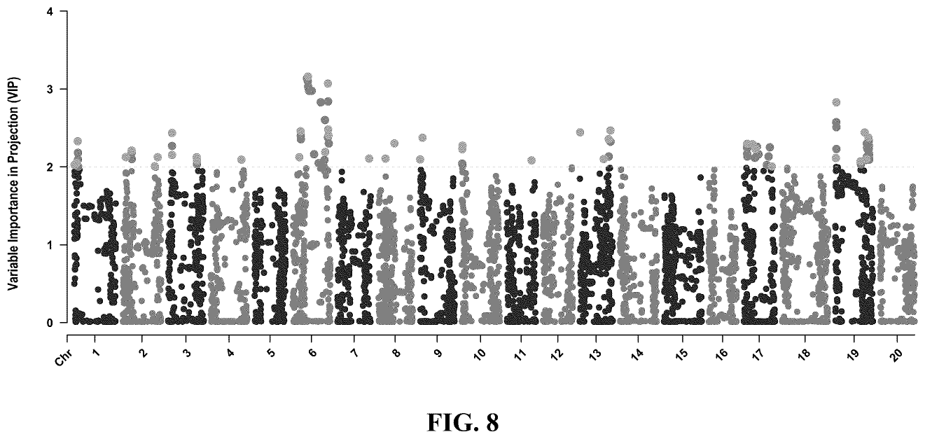

Significant marker-trait associations (logarithm of the odds (LOD)>4.0) were identified using both FarmCPU and BLINK models across chromosomes 6 (LG C2), 8 (LG A2), 9 (LG K), 10 (LG O), and 19 (LG L) (FIGS. 7A and 7B). The genomic regions and harboring candidate genes were reported based on the soybean assembly Williams 82 Version 2 (Genome Browser Wm82.a2, www.soybase.org). In chromosome 6, three separate genomic regions were detected at 10,891,060 bp, 20,739,900 bp, and 47,550,354 bp. The genomic region on chromosome 6 (10,891,060 bp) represented by the SNP ss715592728 (minor allele frequency (MAF) of 0.33) resulted in LOD scores of 5.4 and 12.3 for the FarmCPU and BLINK models, respectively (Table 3). The SNP ss715593866 (MAF of 0.47, 20,739,900 bp) had the highest LOD scores in both FarmCPU and BLINK models (19.8 and 30.3, respectively) across the entire set of SNPs. A Universal Stress Protein (Glyma.06g209600) has been reported within 50 kb of ss715593866. Lastly, ss715594836 (MAF of 0.34, 47,550,354 bp) is co-localized with a glycosyltransferase protein (Glyma.06g286500) and resulted in LOD scores of 6.0 and 7.5 for the FarmCPU and BLINK models, respectively. In chromosome 8, a genomic region at 22,622,648 bp (ss715600920, MAF of 0.17) resulted in LOD scores of 6.1 and 4.3 for the FarmCPU and BLINK models, respectively. A gene (Glyma.08g255800) expressing an S-adenosylmethionine decarboxylase is located within 50 kb of ss715600920. In chromosome 9, ss715604850 (MAF of 0.16, 48,055,288 bp) had LOD scores of 4.9 and 6.4 for the FarmCPU and BLINK models, respectively. Interestingly, an additional glycosyltransferase protein (Glyma.09g224800) is located within 50 kb of ss715604850. The genomic region identified on chromosome 10 (981,062 bp) is co-localized with the region previously reported in Example 1. The SNP ss715608720 (MAF of 0.40) had LOD scores of 4.6 and 6.3 for the FarmCPU and BLINK models, respectively. Two genes with plant herbicide detoxification functions were detected within 50 kb of ss715608720, including Glyma.10g010000 (glycosyltransferase protein) and Glyma.10g010700 (oxidoreductase activity). Lastly, a novel genomic region in chromosome 19 was identified at 1,656,743 bp. The SNP ss715633252 (MAF of 0.47) had LOD scores of 7.5 and 10.0 for the FarmCPU and BLINK models, respectively. Two ATP-binding cassette (ABC) transporter family proteins (Glyma.19g016400 and Glyma.19g016600) were identified within 50 kb of this SNP. A second genomic region at 45,152,186 bp of chromosome 19 was also detected. This region was previously reported in Example 1 and is rich in UDP-dependent glycosyltransferase genes. The SNP ss715635454 (MAF of 0.33) had LOD scores of 6.3 and 13.9 for the FarmCPU and BLINK models, respectively. Across all significant marker-trait associations, the reported candidate genes have biological functions directly associated with the multi-phase herbicide detoxification model.

The genomic regions on chromosomes 6, 8, 9, and 19 identified in this study have not been previously reported as associated with off-target dicamba response.

TABLE 3
Summary of significant marker-trait associations identified using the BLINK and
FarmCPU models including genomic position, minor allele frequency, logarithm
of odds, variable importance in projection, and co-localized candidate genes.

| | | | | LOD³ | | | | |
|---|---|---|---|---|---|---|---|---|
| | | Position | MAF | | Farm | | Candidate | |
| SNP | Chr. | (bp)¹ | (%)² | BLINK | CPU | VIP ⁴ | Genes⁵ | Function⁵ |
| ss715592728 | 6 | 10,891,060 | 0.33 | 12.3 | 5.4 | 2.46 | | |
| ss715593866 | 6 | 20,739,900 | 0.47 | 30.3 | 19.8 | 3.16 | Glyma.06g209600 | Universal Stress |
| | | | | | | | | Protein |
| ss715594836 | 6 | 47,550,354 | 0.34 | 7.5 | 6.0 | 3.07 | Glyma.06g286500 | Glycosyltransferase |
| ss715600920 | 8 | 22,622,648 | 0.17 | 4.3 | 6.1 | 2.30 | Glyma.08g255800 | S- |
| | | | | | | | | adenosylmethionine |
| | | | | | | | | decarboxylase |
| ss715604850 | 9 | 44,855,340 | 0.16 | 6.4 | 4.9 | 1.85 | Glyma.09g224800 | Glycosyltransferase |
| ss715608720 | 10 | 981,062 | 0.40 | 6.3 | 4.6 | 2.23 | Glyma.10g010000 | Glycosyltransferase |
| | | | | | | | Glyma.10g010700 | Oxidoreductase |
| ss715633252 | 19 | 1,656,743 | 0.47 | 10.0 | 7.5 | 2.83 | Glyma.19g016400 | ABC Transporter |
| | | | | | | | | Protein |
| | | | | | | | Glyma.19g016600 | ABC Transporter |
| | | | | | | | | Protein |
| ss715635454 | 19 | 45,152,186 | 0.33 | 13.9 | 6.3 | 2.27 | Glyma.19g187400 | UDP- |
| | | | | | | | | glycosyltransferase |
| | | | | | | | | genes |

¹Position in the genome reported as base pairs (Genome assembly version Wm82.a2).
²Minor allele frequency reported in percentage.
³LOD, the logarithm of odds calculated as the negative logarithm of the observed p-value for each model.
VIP, variable importance in projection.
⁵Candidate Genes and Functions identified within a 50 kb window from the significant SNP (Genome Browser Wm82.a2, www.soybase.org).

### Variable Importance in Projection and Classification Metrics

The distribution of SNPs across chromosomes was relatively uniform with an average of 248 SNPs per chromosome, ranging from 190 (chromosome 17, LG D2) to 327 SNPs (chromosome 8). The average VIP score across 4,970 SNPs was 0.82, ranging from 0.01 (ss715598194) to 3.16 (ss715593866) (FIG. 8). Within chromosomes, the average VIP score ranged from 0.59 (chromosome 8) to 1.02 (chromosome 19). The VIP metric ranks predictors (SNPs) based on their significance to the aggregate index (Dₑ). Given the average of squared VIP scores are equal to 1.0, a threshold higher than 1.0 is employed to select features that make the most substantial contribution to Dₑ. In scenarios where the number of independent variables significantly exceeds the number of observations and there is considerable multicollinearity, a threshold of 2.0 is suggested to filter significant predictors. A total of 113 SNPs with VIP scores above 2.0 were distributed across chromosomes 1 (7 SNPs, LG D1a), 2 (6 SNPs, LG D1b), 3 (6 SNPs, LG N), 4 (1 SNP, C1), 6 (25 SNPs), 7 (1 SNP, LG M), 8 (2 SNPs), 9 (2 SNPs), 10 (4 SNPs), 11 (1 SNP, LG B1), 13 (6 SNPs, LG F), 17 (16 SNPs), and 19 (36 SNPs) (FIG. 8). To further reduce model overfitting, SNPs with absolute values of pairwise Pearson's correlation≥0.7 were removed, resulting in 41 SNPs selected to be included in the ML algorithms. These SNPs were distributed across chromosomes 1 (3 SNPs), 2 (4 SNPs), 3 (4 SNPs), 4 (1 SNP), 6, (7 SNPs), 7 (1 SNP), 8 (2 SNPs), 9 (2 SNPs), 10 (2 SNPs), 11 (1 SNP), 13 (4 SNPs), 17 (4 SNPs), and 19 (6 SNPs) (FIG. 8).

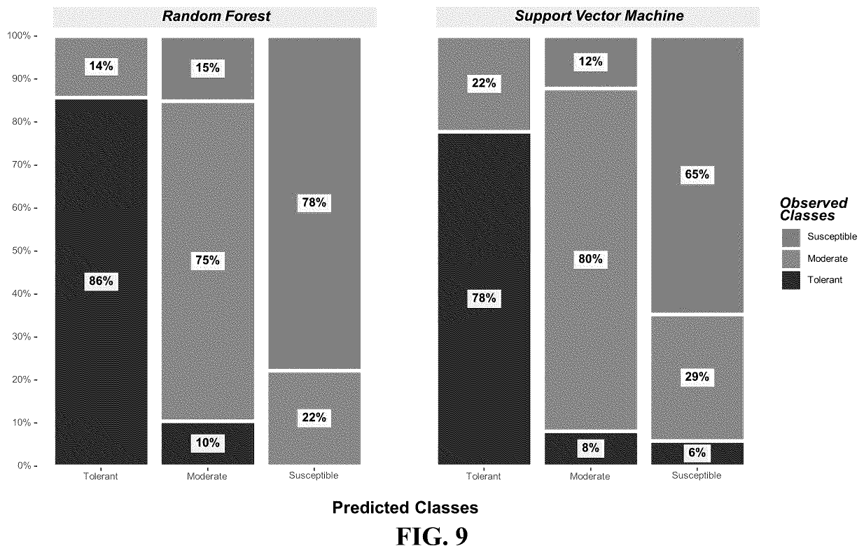

The SVM model yielded the highest overall classification accuracy (0.79) including 12 SNPs as predictors, with a noticeable reduction in overall classification accuracy with the inclusion of more SNPs, The SNPs that resulted in the highest classification accuracy, sorted by order of inclusion in the model, were ss715593866, ss715600920, ss715594836, ss715592728, ss715635403, ss715627948, ss715579081, ss715588076, ss715582179, ss715608720, ss715586851, ss715634898, and ss715616396. The model including the 12 SNPs as predictors outperformed both model including only the highest VIP SNP (ss715593866) and the model including all 41 selected SNPs by approximately 11% (0.71 to 0.79). All classification metrics, including precision and specificity, observed equivalent improvements. The SVM model resulted in minor extreme misclassifications (observed tolerant predicted as susceptible, and vice-versa) highlighting its high suitability to be implemented in an applied soybean breeding pipeline aiming to identify genotypes tolerant to off-target dicamba (FIG. 9). For instance, out of all tolerant predictions, 78% were observed as tolerant and 22% as moderate, while out of all susceptible predictions, 65% were observed as susceptible, 29% as moderate, and only 6% as tolerant (FIG. 9).

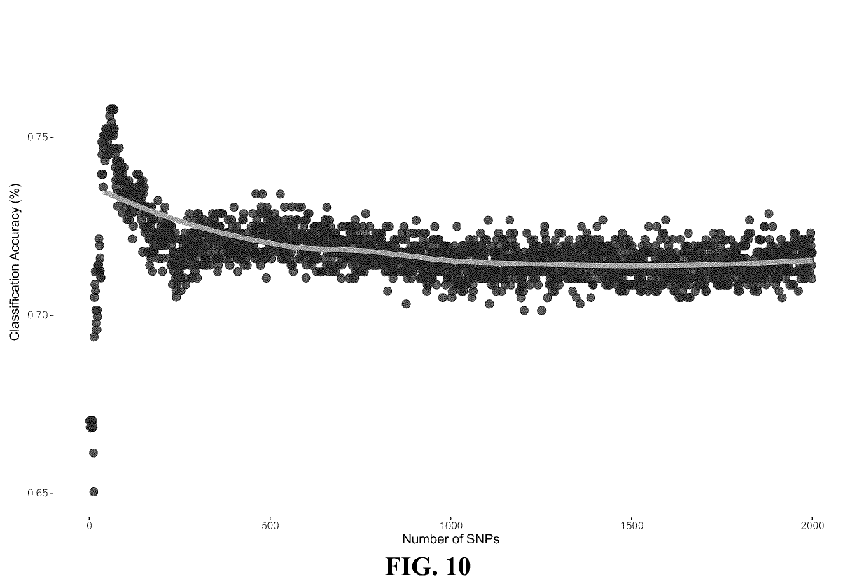

The highest overall classification accuracy (0.76) in the RF model was achieved using 17 SNPs as predictors, including ss715593866, ss715635403, ss715588076, ss715592728, ss715600920, ss715582179, ss715633252, ss715626266, ss715583058, ss715582533, ss715610029, ss715605561, ss715605251, ss715599209, ss715627948, ss715616396, ss715595654, and ss715580115. Eight SNPs, including ss715593866, ss715635403, ss715588076, ss715592728, ss715600920, ss715582179, ss715627948, and ss715616396 overlapped between the SVM and RF models yielding the highest overall classification accuracy. A larger increase in overall classification accuracy (17%) was observed between the baseline model including only ss715593866 (0.65) and the model including 17 SNPs (0.76). Substantial improvements in class accuracy, precision, and specificity were also observed between the two models. The RF model also demonstrated high suitability to be implemented in real-world prediction problems. Out of all tolerant predictions, 86% were observed as tolerant and 14% as moderate, while out of all susceptible predictions, 78% were observed as susceptible and 22% as moderate (FIG. 9). Overall, the RF model did not perform any extreme misclassifications. Similar to the SVM model, a substantial decrease in overall classification accuracy was observed with the inclusion of more predictors (FIG. 10). The overall classification accuracy was computed for each iteration from 1 SNP to 2,000 SNPs. A pronounced negative trend was observed with the increase in SNPs, indicating the negative impact of overfitting and the importance of filtering SNPs on overall model performance (FIG. 10)

### Discussion

The goal of this study was to detect genomic regions related to various responses to prolonged off-target dicamba exposure in a population consisting of advanced soybean breeding lines. A total of 551 non-DT advanced breeding lines derived from 232 unique bi-parental populations were grown in environments surrounded by DT soybean and cotton growing systems, thus being exposed to prolonged unintentional off-target dicamba.

A total of eight genomic regions related to various responses to off-target dicamba were identified across chromosomes 6 (3), 8 (1), 9 (1), 10 (1), and 19 (2). Interestingly, several candidate genes co-localized with significant SNPs have been reported to have biological functions directly related to proteins participating in the three phases of herbicide detoxification in plants. Thus, it can be hypothesized that non-DT soybean genotypes with tolerance response to off-target dicamba may have the capability to more rapidly detoxify low doses of the herbicide compared to sensitive genotypes. For instance, the gene Glyma.06g209600 is located within 50 kb of ss715593866 (LOD scores of 19.8 and 30.3 in the FarmCPU and BLINK models, respectively). This gene has been reported to be a Universal Stress Protein with adenine nucleotide alpha hydrolase function. Phase I of herbicide detoxification usually introduces a reactive functional group for the subsequent metabolism and detoxification through oxidation or hydrolysis by cytochrome P450s or carboxylesterases, respectively. Although the genetic architecture of tolerance is complex and regulated by multiple small and large effect loci, ss715593866 is a major effect SNP and resulted in high classification accuracies in both RF and SVM when included as the sole predictor. Therefore, further investigation of the role and effect of ss715593866 could better explain the physiological mechanisms associated with tolerance to off-target dicamba in soybean.

Glyma.06g286500 is a candidate gene located within 50 kb of ss715594836 (LOD scores of 6.0 and 7.5 in the FarmCPU and BLINK models, respectively) with glycosyltransferase-related functions. Phase II of herbicide detoxification involves conjugation reactions of herbicides with reduced glutathione [catalyzed by glutathione S-transferases (GST)] or glucose (catalyzed by UDP-dependent glycosyltransferases). In chromosome 8, the candidate gene Glyma.08g255800 located within 50 kb of ss715600920 (LOD scores of 6.1 and 4.3 for the FarmCPU and BLINK models, respectively) expresses an S-adenosylmethionine decarboxylase. This enzyme is key in the biosynthesis of polyamines. Although the precise role of S-adenosylmethionine decarboxylase in plants is still unknown, its up-regulation has been reported in response to many abiotic stressors including salt, drought, temperature, and oxidative stress. A consequence of exposure to auxinic herbicides is the rapid increase in ethylene production by initiating 1-aminocyclopropane-1-carboxylic acid synthase and biosynthesis of abscisic acid. This reduces transpiration, carbon dioxide assimilation, starch formation, and a substantial accumulation of reactive oxygen species, which leads to chloroplast damage, membrane destruction, and ultimately tissue damage and cell death.

Similar to Glyma.06g286500, the candidate genes Glyma.09g224800 (co-localized with ss715604850, LOD scores of 4.9 and 6.4 for the FarmCPU and BLINK models, respectively) and Glyma.10g010000 (co-localized with ss715608720, LOD scores of 4.6 and 6.3 for the FarmCPU and BLINK model, respectively) have glycosyltransferase-related functions which are associated with conjugation reactions of phase II of herbicide detoxification. Within the same genomic region of chromosome 10, ss715608720 is also co-localized with Glyma.10g010700, a candidate gene involved in oxidoreductase activity. The expression of oxydoreductase enzymes acts as a signal to the subsequential expression of GST, cytochrome P450 monooxygenases, and other proteins involved in herbicide detoxification. This genomic region was previously reported, and the candidate gene Glyma10g01700, which encodes a multidrug resistance protein (MRP), was co-localized with the significant SNP ss715605561, identified in Example 1. On chromosome 19, a genomic region around 1,650,000 bp (ss715633252, LOD scores of 7.5 and 10.0 for the FarmCPU and BLINK models, respectively) harbors two candidate genes (Glyma.19g016400 and Glyma.19g016600) that belong to the ABC transporter family. Herbicide conjugates from phase II are transported into the vacuole of plant cells by transporters, concluding phase III of herbicide detoxification. Another genomic region on chromosome 19 around 45,000,000 bp was detected and previously reported in Example 1. This genomic region contains several UDP-glycosyltransferase genes which are necessary for phase II reactions of herbicide detoxification.

One of the main challenges in analyzing high-dimensional genomic data is the presence of multicollinearity and excessive noise among predictors, which often leads to a substantial detection of false-positive associations in GWAS. Given the substantial imbalance between the number of predictors (SNPs) and observations, traditional GWAS models frequently face the risk of overfitting. In this scenario, the model overly captures unintended noise in the training set, yielding low reproducibility on the testing set. An approach to avoid overfitting and improve model reproducibility and cost-effectiveness is feature selection, which is the process of selecting relevant predictors from the original predictors set. In this study, a supervised feature dimension reduction based on VIP scores initially selected predictors with high importance toward the aggregate index (Dₑ). This was followed by a pair-wise correlation filtering step, resulting in a subset of important, uncorrelated SNPs. In both RF and SVM models, a pronounced decrease in prediction accuracy was observed with the increment of SNPs as predictors. Therefore, identifying fewer but relevant predictors (i.e. feature selection) yielded higher prediction accuracies as compared to fitting the model with the highest number of predictors available. In this study, less than 0.5% of total predictors were needed to achieve the highest prediction accuracy in both RF (17 out of 4,970 SNPs) and SVM (12 out of 4,970 SNPs) models. Therefore, the combination of feature selection and predictive classification algorithms may provide high accuracies in the identification and selection of genotypes with desirable phenotypes for both qualitative and quantitative traits.

Both RF and SVM models yielded high classification accuracies using the best combination of predictors (0.76 and 0.79, respectively). Both prediction models resulted in high precision, meaning that minimal extreme misclassifications (observed tolerant predicted as susceptible, and vice-versa) were observed. Visual assessment of off-target dicamba tolerance is directly associated with seed yield under prolonged off-target dicamba exposure. On average, a yield penalty of 8.8% (confidence interval of 7.0 to 10.6%) has been observed for each unit increase in damage score on a similar 1-4 scale. Therefore, the identification and development of non-DT soybean genotypes with superior tolerance to off-target dicamba can help sustain the production of non-DT herbicide-tolerance systems, which currently represent nearly 14.2 million hectares. In addition, natural tolerance may improve the sustainability of niche markets for food-graded non-GMO soybean. Genomic prediction models, such as those reported in this study, can significantly speed up the identification of genotypes with superior tolerance to off-target dicamba. The understanding of the genetics and physiological mechanisms underlying the differential responses to off-target dicamba is critical to support soybean breeding programs focusing on the development of non-DT soybean genotypes with superior tolerance to off-target dicamba.

The SNPs identified and deemed significant in Examples 1 and 2 are listed in Table 4. Of these, the SNPs on Chromosome 6 (ss715593866), 10 (ss715605561), and 19 (ss715635349) contributed the most to the variation in response to dicamba. The others, although significant, provided a minor contribution to the variation in response to dicamba.

TABLE 4

| | Chromosome; | |
|---|---|---|
| | position | |
| | (bp) based | |
| | on Genome | Genomic |
| | assembly | DNA Sequence |
| | version | SNP site |
| Marker Name | Wm82.a2 | is underlined |
| ss715635349 | 19; | TGGTGGGTGGTGACAATGAATCAGGA |
| (SEQ ID | 44,580,800 | ACCCAATTGGTGGATTGTGGTGTGGA |
| NO: 1) | | AGATGCAATGAAGCTTTCTATCAGTG |
| | | TCCCCACAGCATCGCATCCCCACAAC |
| | | GGTAGATCTAGATCTGG |
| ss715605561 | 10; | CTCTCAGAGATGGGTAACCAATTAGT |
| (SEQ ID | 1,227,933 | CTCACTACGCCCCTCAGAAGCCACGG |
| NO: 2) | | GCAAAATTATTACGTGGGATGGTTCG |
| | | GTGCACGAAATTGAGCAATCTTTGAC |
| | | AGTGGCAGAGCTGATGC |
| ss715609879 | 11; | ATTAGATGCCTAAGTAGTGTCATTTA |
| (SEQ ID | 15,740,804 | GATCTTTAACCTTCATTGTAGTGTTG |
| NO: 3) | | CATATTATTTTTCCATTGTAATGATA |
| | | CAATCCTACCTTCTAAGGGCATTGGA |
| | | TAGAAGACTCCAAGAAG |
| ss715632413 | 18; | ACACATTAACTTTTTATGATTTATTT |
| (SEQ ID | 57,025,570 | GATAATATATATGTTTGAGTTAATGC |
| NO: 4) | | TGAGTTTAAAATCAGATGGATCCTAA |
| | | GTATTTATGGCTTTCTATCATTCTCT |
| | | GAAGCATTCCTGTTGCA |
| ss715622838 | 15; | TAGGAGTGTTCGTACTTCTTCCTTAG |
| (SEQ ID | 5,457,236 | ACTAGTGCACATGAATTAAGGATTAA |
| NO: 5) | | TGCTTAACGGATCGAGATAATTTAAA |
| | | CTAAAAAAATTACAGTATCTTTTTTT |
| | | TAATATCTCATTATTAA |
| ss715619759 | 14; | AAAATGATTTTTGTGTTATATCACAA |
| (SEQ ID | 6,460,927 | AACTTCAAGGAGTGTTAGTTTAGTTT |
| NO: 6) | | CTTTTTTTTAAGGAACTTAGTTTACT |
| | | CTTTTAAATATTATAAGATGCTCTCT |
| | | CTTTAGACTCAAAGGTA |
| ss715590768 | 5; | GAATCATCACAAGTTACTGAACACTA |
| (SEQ ID | 32,594,828 | GAGTGAATAATATTTTAAATGAAATG |
| NO: 7) | | ATCCTAATTCGCATCACTTATGAGTC |
| | | ACTTGACGATAACTTAACCCTTAATC |
| | | TCGACTAAGAAACTTAT |
| ss715592527 | 5; | ATATCCAAAAAATCAATAGCTTTTTG |
| (SEQ ID | 2,516,484 | ATAAACCATTTCTTCGTTCATCCAAA |
| NO: 8) | | CAGGATGTCATAATATTTATCTAGTT |
| | | CTTCATTTGCTGATATGCATTTGGCA |
| | | GAATAATTGTCTTCTTT |
| ss715632432 | 18; | GGAGAAGACATTGATCCTTCTTGTTA |
| (SEQ ID | 57,206,151 | CGAACAAGATACCGCGGGTCTTTCAG |
| NO: 9) | | AAGAGGAAGTGGAAGAGATTAGAAGG |
| | | CTTCATGCAAGTGACACGGCCATTGA |
| | | CAAAGAGAAAGACTCAA |
| ss715632412 | 18; | ATAAAAACCATGCACTTAAATTGGGT |
| (SEQ ID | 57,013,050 | GCATCACGTGAGTAGCCCTTTTTTTC |
| NO: 10) | | AGAGAGGACCAAATATAAATATTAAA |
| | | TATATAAAATTCAACTAATAATTAAA |
| | | AGACTTAGTGGTTAATG |
| ss715592728 | 6; | AGTGATTGTCCGACTAAGATAGAGTT |
| (SEQ ID | 10,891,060 | GACTTGACATATTTTACTTTTTATTC |
| NO: 11) | | CAATTTCTTTTACTTTTTTATAAATT |
| | | GTATTCCTAATTTTTTTATATTTTGG |
| | | CATATTAAACAATGACA |
| ss715593866 | 6; | TTCTATCTAAACTAACCAAACTCCCA |
| (SEQ ID | 20,739,900 | TTGCTAGCCGACCTTAGAGCATAGTT |
| NO: 12) | | ATCAAATTTGATTTGGACCAATCAGG |
| | | TCGACCCATTGAACCGAGAACTGGGG |
| | | CACCAAATTGGCTCAAA |
| ss715594836 | 6; | AAAGGTCACAATTATTTCGATAAATA |
| (SEQ ID | 47,550,354 | TACCTCTGACTGAATAGGTGGTAGGA |
| NO: 13) | | AATCATGGAAAACATATACAATGAAT |
| | | AAAGGATAGCAAGACAATACAAAGTG |
| | | ATAGAAAGAGATCAAAC |
| ss715600920 | 8; | CAGAAAAAAAGATTAAGCTATGTTGA |
| (SEQ ID | 22,622,648 | CCCACAAAAATGCCAAATACATGATA |
| NO: 14) | | CTGGAAAGACTTTAACTCTCCTTCTC |
| | | TGGTTCTATTGTTTGTTGCGTCATAG |
| | | TTCTTGTTGGTGTATGC |
| ss715604850 | 9; | ACCACAACAACTTGACTTGATAAAAG |
| (SEQ ID | 44,855,340 | AATGGCAAATGCATGAAAACATCAAT |
| NO: 15) | | TTTTGTAACTGTACCGTTCACAATTT |
| | | CTTTATTGGAAACAATATGGAATAAA |
| | | GTAGACCATGAATGCAG |
| ss715608720 | 10; | CAAACCAAAGTGGACTTGTGTTACAC |
| (SEQ ID | 981,062 | GCTTCATCATCACTGCTTCCTTCGAA |
| NO: 16) | | TTCCGACCATAGTATTGAAAGGGGGT |
| | | CGGATCTGTGGCCAGGTTTCAATAAC |
| | | CAAATGTTGTTGGAACA |
| ss715633252 | 19; | TTAGACCCTGCTAAAGAACAAAGACT |
| (SEQ ID | 1,656,743 | CCATCGGTGTTGGGTTGCAGCTGCGA |
| NO: 17) | | GACCAAAGTGTAAGGGACGTCTTTAC |
| | | AGTATTGGAGACCTTGCTCATATTTA |
| | | TAAATGTGGAGATGAGA |
| ss715635454 | 19; | TACATTCTCATTTGCAAGCTTGGACA |
| (SEQ ID | 45,152,186 | AGGCAGAGACATATGATCCTAAGTGG |
| NO: 18) | | GTAAATGCACAGGACCTCTGTGCCCA |
| | | AAAGGGTTCAAAACTTCAAGGTGGGG |
| | | TTGGACCATTTGGGCTT |

What is claimed is:

1. A method of producing a soybean plant having at least one allele associated with dicamba tolerance, the method comprising:
providing a first soybean plant comprising at least one molecular marker associated with dicamba tolerance;
providing a second soybean plant;
crossing the first soybean plant with the second soybean plant to produce a population of soybean progeny plants; and
selecting from said population of soybean progeny plants a soybean plant having at least one allele associated with dicamba tolerance.

2. The method of claim 1, wherein the molecular marker is one or more of ss715635349, ss715605561, ss715593866, or a marker within 1 cM, 2 cM, or 5 cM thereof.

3. The method of claim 1, wherein the molecular marker is a thymine (T) at position 61 of SEQ ID NO: 1, an adenine (A) at position 61 of SEQ ID NO: 2, and/or a T at position 61 of SEQ ID NO: 12.

4. The method of claim 1, wherein the crossing step further comprises one or more backcrosses.

5. The soybean plant or a plant part thereof obtained by the method of claim 1, wherein the plant or plant part comprises at least one allele associated with dicamba tolerance.

6. The soybean plant or plant part of claim 5, wherein the plant or plant part exhibits increased tolerance to dicamba as compared to a control soybean plant lacking the at least one allele associated with dicamba tolerance.

7. A method of identifying and/or selecting a soybean plant having at least one allele associated with dicamba tolerance, the method comprising:
a. isolating a nucleic acid from a soybean plant;
b. detecting in the nucleic acid the presence of at least one molecular marker associated with dicamba tolerance; and
c. identifying and/or selecting a soybean plant based on the presence of at least one molecular marker associated with dicamba tolerance.

8. The method of claim 7, wherein the molecular marker is one or more of ss715635349, ss715605561, ss715593866, or a marker within 1 cM, 2 cM, or 5 cM thereof.

9. The method of claim 7, wherein the molecular marker is a thymine (T) at position 61 of SEQ ID NO: 1, an adenine (A) at position 61 of SEQ ID NO: 2, and/or a T at position 61 of SEQ ID NO: 12.

10. The method of claim 7, wherein the identified and/or selected soybean plant exhibits increased tolerance to dicamba as compared to a control soybean plant lacking the at least one allele associated with dicamba tolerance.

11. The method of claim 7, wherein the soybean plant from step a) and/or the soybean plant identified and/or selected in step c) is a soybean plant from a population generated by a cross of a soybean plant having increased dicamba tolerance and a dicamba sensitive soybean plant.

12. A method of producing a soybean plant having at least one allele associated with dicamba tolerance, the method comprising:
a. crossing the soybean plant identified and/or selected in step c) of claim 7 with a second soybean plant to produce a population of progeny soybean plants; and
b. selecting from said population of soybean progeny plants a soybean plant having at least one allele associated with dicamba tolerance.

13. The soybean plant or a plant part thereof selected in step b) of claim 12.

14. A method of identifying and/or selecting a soybean plant having increased tolerance to dicamba, the method comprising:
a. screening a population of soybean plants with a molecular marker to determine if one or more soybean plants from the population comprises at least one allele associated with dicamba tolerance; and
b. selecting and/or identifying from said population at least one soybean plant comprising the at least one allele associated with dicamba tolerance.

15. The method of claim 14, wherein the molecular marker is one or more of ss715635349, ss715605561, ss715593866, or a marker within 1 cM, 2 cM, or 5 cM thereof.

16. The method of claim 14, wherein the molecular marker is a thymine (T) at position 61 of SEQ ID NO: 1, an adenine (A) at position 61 of SEQ ID NO: 2, and/or a T at position 61 of SEQ ID NO: 12.

17. The method of claim 14, wherein the population is generated by a cross of a soybean plant having increased dicamba tolerance and a dicamba sensitive soybean plant.

18. The method of claim 14, further comprising:
c. crossing the soybean plant selected and/or identified in step b) to a second soybean plant; and
d. obtaining a progeny soybean plant.

19. The progeny soybean plant or a plant part thereof obtained by the method of claim 18, wherein the plant or plant part comprises at least one allele associated with dicamba tolerance.

20. The progeny soybean plant or plant part of claim 19, wherein the plant or plant part exhibits increased tolerance to dicamba as compared to a control soybean plant lacking the at least one allele associated with dicamba tolerance.
